SEQUENCE LISTING (50 sequences)

SEQ ID NO: 1 - protein, 8 aa, Artificial Sequence
  Heavy chain variable region CDR1
GFNIKDTF

SEQ ID NO: 2 - protein, 8 aa, Artificial Sequence
  Heavy chain variable region CDR2
IDPANTNT

SEQ ID NO: 3 - protein, 11 aa, Artificial Sequence
  Heavy chain variable region CDR3
VRGLYTYYFDY

SEQ ID NO: 4 - protein, 118 aa, Artificial Sequence
  Variable heavy chain
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSS

SEQ ID NO: 5 - protein, 6 aa, Artificial Sequence
  Variable light chain CDR1
QSISNN

SEQ ID NO: 6 - protein, 9 aa, Artificial Sequence
  Variable light chain CDR1
QQGNSWPYT

SEQ ID NO: 7 - protein, 107 aa, Artificial Sequence
  Variable light chain
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIK

SEQ ID NO: 8 - protein, 8 aa, Artificial Sequence
  Variable heavy chain CDR1
GFNIKDTH

SEQ ID NO: 9 - protein, 8 aa, Artificial Sequence
  Variable heavy chain CDR2
IDPANGNT

SEQ ID NO: 10 - protein, 11 aa, Artificial Sequence
  Variable heavy chain CDR3
ARWGTNVYFAY

SEQ ID NO: 11 - protein, 118 aa, Artificial Sequence
  Variable heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSS

SEQ ID NO: 12 - protein, 5 aa, Artificial Sequence
  Variable light chain CDR1
SSVSY

SEQ ID NO: 13 - protein, 9 aa, Artificial Sequence
  Variable light chain CDR3
QQYHSYPPT

SEQ ID NO: 14 - protein, 106 aa, Artificial Sequence
  Variable light chain
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIK

SEQ ID NO: 15 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, Artificial Sequence
  Modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 17 - protein, 448 aa, Artificial Sequence
  Modified Fc region
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 18 - protein, 447 aa, Artificial Sequence
  Modified Fc region
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 19 - protein, 214 aa, Artificial Sequence
  Light chain
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 448 aa, Artificial Sequence
  Heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 21 - protein, 447 aa, Artificial Sequence
  Heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 22 - protein, 213 aa, Artificial Sequence
  Light chain
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 118 aa, Artificial Sequence
  Variable heavy chain
EVQLQQSGAEFVKPGASVKLSCTASGFNIKDTFIHWVKQRPEQGLDWIGRIDPANGNTKYDPKFQGKATETTDTSSNTAYLQLSSLTSEDTAVYYCVRGLYTYYFDYWGQGTSVTVSS

SEQ ID NO: 24 - protein, 107 aa, Artificial Sequence
  Variable light chain
DIVLTQSPATLSVTPGDSVSLSCRASQSISNNLHWYQQKSHESPRLLIKFASQSISGIPSRFSGSGSGADFTLIINSVETEDFGMYFCQQGNSWPYTFGGGTKLEIK

SEQ ID NO: 25 - protein, 448 aa, Artificial Sequence
  Heavy chain
EVQLQQSGAEFVKPGASVKLSCTASGFNIKDTFIHWVKQRPEQGLDWIGRIDPANGNTKYDPKFQGKATETTDTSSNTAYLQLSSLTSEDTAVYYCVRGLYTYYFDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHGALHNHYTQKSLSLSPGK

SEQ ID NO: 26 - protein, 214 aa, Artificial Sequence
  Light chain
DIVLTQSPATLSVTPGDSVSLSCRASQSISNNLHWYQQKSHESPRLLIKFASQSISGIPSRFSGSGSGADFTLIINSVETEDFGMYFCQQGNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 118 aa, Artificial Sequence
  Variable heavy chain
EVQLQQSGAELVKPGASVKLFCTASGFNIKDTHIHWVKQRPEQGLEWIGRIDPANGNTEYDPKFQGKATIRVDTSSDTAYLQLSSLTSEDTAVHYCARWGTNVYFAYWGQGTSVTVSS

SEQ ID NO: 28 - protein, 106 aa, Artificial Sequence
  Variable light chain
DIQLTQSPAIMSASPGEKVTISCSASSSVSYMYWYQQKPGSSPKPWIYRTSNLASGVPGRFSGSGSGTSYSLTISSMEAEDAATYYCQQYHSYPPTFGGGTKLEIK

SEQ ID NO: 29 - protein, 448 aa, Artificial Sequence
  Variable heavy chain
EVQLQQSGAELVKPGASVKLFCTASGFNIKDTHIHWVKQRPEQGLEWIGRIDPANGNTEYDPKFQGKATIRVDTSSDTAYLQLSSLTSEDTAVHYCARWGTNVYFAYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHGALHNHYTQKSLSLSPGK

SEQ ID NO: 30 - protein, 213 aa, Artificial Sequence
  Light chain
DIQLTQSPAIMSASPGEKVTISCSASSSVSYMYWYQQKPGSSPKPWIYRTSNLASGVPGRFSGSGSGTSYSLTISSMEAEDAATYYCQQYHSYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 8 aa, Artificial Sequence
  Variable heavy chain CDR1
GYRFSNFV

SEQ ID NO: 32 - protein, 8 aa, Artificial Sequence
  Variable heavy chain CDR2
INPYNGNK

SEQ ID NO: 33 - protein, 20 aa, Artificial Sequence
  Variable heavy chain CDR3
ARVGPYSWDDSPQDNYYMDV

SEQ ID NO: 34 - protein, 127 aa, Artificial Sequence
  Variable heavy chain
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 35 - protein, 7 aa, Artificial Sequence
  Variable light chain CDR1
HSIRSRR

SEQ ID NO: 36 - protein, 9 aa, Artificial Sequence
  Variable light chain CDR3
QVYGASSYT

SEQ ID NO: 37 - protein, 108 aa, Artificial Sequence
  Variable light chain
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 38 - protein, 457 aa, Artificial Sequence
  Heavy chain
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 215 aa, Artificial Sequence
  Light chain
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 457 aa, Artificial Sequence
  Heavy Chain
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 41 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 42 - protein, 440 aa, Homo sapiens [sequence omitted]

SEQ ID NO: 43 - protein, 411 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 44 - protein, 411 aa, Artificial Sequence
  Modified DR5 protein
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLANQKIEDHLLSSGKFMYLEGNADSAMS

SEQ ID NO: 45 - protein, 445 aa, Macaca fascicularis
MGQLRQSAPAASGARKGRGPGPREARGARPGLRVLKTLVLVVAAARVLLSVSADCAPITRQSLDPQRRAAPQQKRSSPTEGLCPPGHHISEDSRECISCKYGQDYSTHWNDFLFCLRCTKCDSGEVEVNSCTTTRNTVCQCEEGTFREEDSPEICRKCRTGCPRGMVKVKDCTPWSDIECVHKESGTKHTGEVPAVEKTVTTSPGTPASPCSLSGIIIGVIVLVVIVVVAVIVWKTSLWKKVLPYLKGVCSGGGGDPERVDSSSHSPQRPGAEDNALNEIVSIVQPSQVPEQEMEVQEPAEQTDVNTLSPGESEHLLEPAKAEGPQRRGQLVPVNENDPTETLRQCFDDFAAIVPFDAWEPLVRQLGLTNNEIKVAKAEAASSRDTLYVMLIKWVNKTGRAASVNTLLDALETLEERLAKQKIQDRLLSSGKFMYLEDNADSATS

SEQ ID NO: 46 - protein, 416 aa, Artificial Sequence
  Modified DR5 protein
MGQLRQSAPAASGARKGRGPGPREARGARPGLRVLKTLVLVVAAARVLLSVSADCAPITRQSLDPQRRAAPQQKRSSPTEGLCPPGHHISEDSRECISCKYGQDYSTHWNDFLFCLRCTKCDSGEVEVNSCTTTRNTVCQCEEGTFREEDSPEICRKCRTGCPRGMVKVKDCTPWSDIECVHKESGIIIGVIVLVVIVVVAVIVWKTSLWKKVLPYLKGVCSGGGGDPERVDSSSHSPQRPGAEDNALNEIVSIVQPSQVPEQEMEVQEPAEQTDVNTLSPGESEHLLEPAKAEGPQRRGQLVPVNENDPTETLRQCFDDFAAIVPFDAWEPLVRQLGLTNNEIKVAKAEAASSRDTLYVMLIKWVNKTGRAASVNTLLDALETLEERLANQKIQDRLLSSGKFMYLEDNADSATS

SEQ ID NO: 47 - protein, 381 aa, Mus musculus
MEPPGPSTPTASAAARADHYTPGLRPLPKRRLLYSFALLLAVLQAVFVPVTANPAHNRPAGLQRPEESPSRGPCLAGQYLSEGNCKPCREGIDYTSHSNHSLDSCILCTVCKEDKVVETRCNITTNTVCRCKPGTFEDKDSPEICQSCSNCTDGEEELTSCTPRENRKCVSKTAWASWHKLGLWIGLLVPVVLLIGALLVWKTGAWRQWLLCIKRGCERDPESANSVHSSLLDRQTSSTTNDSNHNTEPGKTQKTGKKLLVPVNGNDSADDLKFIFEYCSDIVPFDSWNRLMRQLGLTDNQIQMVKAETLVTREALYQMLLKWRHQTGRSASINHLLDALEAVEERDAMEKIEDYAVKSGRFTYQNAAAQPETGPGGSQCV

SEQ ID NO: 48 - protein, 450 aa, Artificial Sequence
  Modified DR5 protein
MEQRGQNAPAASGARKRHGPGPREARGARPGLRVPKTLVLVVAAVLLLVSAESALITQQDLAPQQRVAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPEA

SEQ ID NO: 49 - protein, 422 aa, Artificial Sequence
  Modified DR5 protein
MEQRGQNAPAASGARKRHGPGPREARGARPGPRVPKTLVLVVAAVLLLVSAESALITQQDLAPQQRAAPQQKRSSPSEGPCLAGQYLSEGNCKPCREGIDYTSHSNHSLDSCILCTRCDSGEVELSPCTTTRNTVCQCEEGTFREEDSPEMCRKCRTGCPRGMVKVGDCTPWSDIECVHKESGAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPE

SEQ ID NO: 50 - protein, 423 aa, Artificial Sequence
  Modifed DR5 protein
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCKPGTFEDKDSPEICQSCSNCTDGEEEVGDCTPWSDIECVHKESGAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPEA

(19) United States
(12) Patent Application Publication
(10) Pub. No.: US 2023/0293680 A1
(43) Pub. Date: Sep. 21, 2023

(54) A COMPOSITION OF ANTI-DR5 ANTIBODIES AND AN IMMUNOMODULATORY IMIDE DRUG FOR USE IN TREATING MULTIPLE MYELOMA

(71) Applicant: Genmab B.V., Utrecht (NL)

(72) Inventors: Hilma VAN DER HORST, Utrecht (NL); Tuna MUTIS, Amsterdam (NL); Kristin STRUMANE, Utrecht (NL); Esther BREIJ, Utrecht (NL); Marije OVERDIJK, Utrecht (NL)

(21) Appl. No.: 18/017,241

(22) PCT Filed: Jul. 23, 2021
(86) PCT No.: PCT/EP2021/070764

§ 371 Date: Jan. 20, 2023

(30) Foreign Application Priority Data
Jul. 23, 2020 (DK) PA 2020 00865

(51) Int. Cl. A61K 39/395; A61K 31/454; A61K 31/69; A61P 35/00

(52) U.S. Cl. CPC A61K 39/3955; A61K 31/454; A61K 31/69; A61P 35/00; A61K 2039/507

(57) ABSTRACT

The present invention relates to the treatment of multiple myeloma using a combination of two antibody molecules that bind to human DR5 antigen and an immunomodulatory imide dmg. The present invention further relates to treatment of relapsed and/or refectory multiple myeloma.

### FIELD OF THE INVENTION

The present invention relates to the treatment of multiple myeloma using a combination of two antibody molecules that bind to human DR5 antigen and an immunomodulatory imide drug. The present invention further relates to treatment of relapsed and/or refectory multiple myeloma.

### BACKGROUND OF THE INVENTION

DR5, also known as death receptor 5, Tumor necrosis factor receptor superfamily member 10B, TNFRSF10B, TNF-related apoptosis-inducing ligand receptor 2, TRAIL receptor 2, TRAIL-R2 and CD262, is a cell surface receptor of the TNF receptor superfamily that binds tumor necrosis factor-related apoptosis-inducing ligand (TRAIL) and mediates apoptosis. In the absence of ligand, DR5 exists in the cell membrane either as monomer or as pre-assembled complexes of two or three receptors through interactions of the first cysteine-rich domain, also known as pre-ligand assembly domain (PLAD).

Based on the sensitivity of cancer cells to TRAIL-mediated apoptosis, numerous agents were developed to activate this pathway to induce apoptosis selectively in cancer cells. A series of conventional (monospecific, bivalent) anti-DR5 antibodies have been developed and tested in the clinic (reviewed in Ashkenazi et al., Nat Rev Drug Discov. 2008 December; 7(12):1001-12; Trivedi et al., Front Oncol. 2015 Apr. 2; 5:69; Yuan et al., Cancer Metastasis Rev 2018 December; 37(4):733-748; Krets et al., Cancers 2019 Mar. 30; 11(4):456). Clinical studies with these compounds demonstrated that DR5 antibodies were generally well tolerated but failed to show convincing and significant clinical benefit.

Multiple myeloma (MM) is a B cell malignancy characterized by the latent accumulation in bone marrow of secretory plasma cells with a low proliferative index and an extended life span. The disease ultimately attacks bones and bone marrow, resulting in multiple tumors and lesions throughout the skeletal system. Approximately 1% of all cancers, and slightly more than 10% of all hematologic malignancies, can be attributed to multiple myeloma. Incidence of MM increases in the aging population, with the median age at time of diagnosis being about 61 years.

Currently available therapies for multiple myeloma include chemotherapy, stem cell transplantation, Darzalex® (daratumumab), Thalomid® (thalidomide), Velcade® (bortezomib), Aredia® (pamidronate), and Zometa® (zoledronic acid). Current treatment protocols, which include a combination of chemotherapeutic agents such as vincristine, BCNU, melphalan, cyclophosphamide, adriamycin, and prednisone or dexamethasone, yield a complete remission rate of only about 5%, and median survival is approximately 36-48 months from the time of diagnosis. Ultimately, all MM patients relapse, even under maintenance therapy with interferon-alpha (IFN-a) alone or in combination with steroids.

A combination of two non-competing anti-DR5 antibodies comprising an Fc region of a human IgG1 and an antigen binding region binding to DR5, wherein the Fc region comprises an E430G mutation, was found to facilitate hexamerization of the antibodies on the cell-surface upon antigen binding and significantly enhances the potency of the antibodies in inducing apoptosis and cell death (PCT/EP2016/079518).

In spite of recent progress in the development of treatment for multiple myeloma, there remains an unmet medical need for patients and anti-DR5 antibodies offer a promising strategy. However, there is a need for enhancing the efficacy of the treatment of patients with multiple myeloma.

### OBJECT OF THE INVENTION

It is an object of the present invention to provide methods for treating multiple myeloma. It is a further object of the present invention to provide a method for treating relapsed and/or refractory multiple myeloma. It is a further object of the present invention to provide a combination of compounds suitable for such use.

### SUMMARY OF THE INVENTION

The present inventors have developed an improved combination treatment comprising two non-overlapping anti-DR5 antibodies and an immunomodulatory imide drug for the treatment of multiple myeloma. Suitable immunomodulatory imide drugs may belong to the class of thalidomide or thalidomide analogues such as lenalidomide. Accordingly, the present invention relates to a first and second anti-DR5 antibody for use in the treatment of multiple myeloma in combination with an immunomodulatory imide drug.

Thus, in one aspect, the invention relates to a method of treating multiple myeloma in a subject, the method comprising administering to a subject in need thereof a first antibody capable of binding DR5 and a second antibody capable of binding DR5, or a pharmaceutically acceptable salt thereof, in combination with an immunomodulatory imide drug.

In one embodiment of the invention, the first antibody comprises a variable heavy chain region and a variable light chain region wherein the variable heavy chain region comprises the CDR1, CDR2 and CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

In one embodiment of the invention, the second antibody comprises a variable heavy chain region and a variable light chain region wherein the variable heavy chain region comprises the CDR1, CDR2 and CDR3 sequences of SEQ ID Nos: 8, 9, and 10 respectively; and wherein the variable light chain region comprises the CDR1, CDR2 and CDR3 sequences of SEQ ID Nos: 12, RTS, and 13, respectively.

In one embodiment of the invention, the first antibody comprises the heavy chain and light chain as set forth in SEQ ID Nos: 18 and 19, respectively.

In one embodiment of the invention, the second antibody comprises the heavy chain and light chain as set forth in SEQ ID Nos: 21 and 22, respectively.

In one embodiment of the invention, the first and second antibody comprises an Fc region of a human IgG1, wherein the Fc region comprises an E430G mutation of an amino acid position corresponding E430 in human IgG1, wherein the amino acid position is according to the Eu numbering.

In one embodiment of the invention, the immunomodulatory imide drug is thalidomide or a thalidomide analog, e.g. lenalidomide or pomalidomide.

In one embodiment of the invention, the immunomodulatory imide drug is lenalidomide.

In a further aspect, the invention relates to a composition comprising a first antibody capable of binding DR5, or a pharmaceutically acceptable salt thereof and second antibody capable of binding DR5, or a pharmaceutically acceptable salt thereof, for use in the treatment of multiple myeloma in combination with an immunomodulatory imide drug.

In another aspect, the invention relates to a first antibody or pharmaceutically acceptable salt thereof, capable of binding DR5, for use in the treatment of multiple myeloma in combination with an immunomodulatory imide drug and a second antibody or pharmaceutically acceptable salt thereof, capable of binding DR5.

In a further aspect, the invention relates to a kit of parts comprising a first antibody capable of binding DR5 and a second antibody capable of binding DR5, or a pharmaceutically acceptable salt thereof, and an immunomodulatory imide drug.

### BRIEF DESCRIPTION OF THE DRAWINGS

FIG. 1: Binding of IgG1-hDR5-01-G56T-E430G and IgG1-hDR5-05-E430G to CHO-S cells transiently transfected to express (A) human and (B) cynomolgus monkey DR5. Antibody binding was tested by flow cytometry and expressed as the geometric mean of the fluorescence intensity (FI) of duplicate samples±standard deviation (SD). IgG1-b12 was used as an isotype control antibody.

FIG. 2: Anti-tumor-activity in cell line derived xenograft (CDX) models in vivo. Evaluation of the in vivo efficacy of different doses of the mixture of IgG1-DR5-01-K409R-E430G+IgG1-DR5-05-F405L-E430G in subcutaneous (SC) cancer xenograft models. IgG1-b12 was used as negative control antibody (isotype control). Tumor size (mean±standard error of mean [SEM]) in mice treated with the indicated antibody dose is shown in time.

FIG. 3: Anti-tumor-activity in patient derived xenograft (PDX) models in vivo. Evaluation of the in vivo efficacy of different doses of the mixture of IgG1-hDR5-01-G56T-E430G+IgG1-hDR5-05-E430G in colorectal cancer PDX models CR0126 and CR3056. IgG1-b12-E430G was used as negative control antibody (isotype control). Tumor size (mean±SEM) in mice treated with the indicated dose is shown in time.

FIG. 4: Plasma concentration-time profiles following a single intravenous (IV) dose of the mixture of IgG1-hDR5-01-G56T-E430G+IgG1-hDR5-05-E430G in female cynomolgus monkeys. Three dose levels were tested with three female monkeys each: (A) 0.5 mg/kg; (B) 5 mg/kg; (C) 25 mg/kg. Post-dose samples were taken at 1, 3, 6, 12, 24 hours, 2, 3, 7, 14, 21, 22, 35, 49 days after dosing. The dotted line indicates the predicted pharmacokinetic (PK) profile of IgG1 using a 2 compartment model, with k10 (clearance constant) at 0.006 h⁻¹, Vc (plasma vol) 40 mL·kg⁻¹ and 5 kg bodyweight.

FIG. 5: Plasma concentration-time profiles following multiple IV doses of the mixture of IgG1-hDR5-01-G56T-E430G+IgG1-hDR5-05-E430G in female cynomolgus monkeys. Four dose groups were tested with two animals each: 0.1 mg/kg, 0.5 mg/kg, 5 mg/kg and 25 mg/kg.

FIG. 6: Plasma concentration-time profiles following once-weekly IV doses of the mixture of IgG1-hDR5-01-G56T-E430G+IgG1-hDR5-05-E430G in male and female cynomolgus monkeys. Four dose groups were tested with five male and five female animals each: 0, 2, 10, and 50 mg/kg. Graphs represent mean plasma concentration-time profiles for IgG1-hDR5-01-G56T-E430G (left) and IgG1-hDR5-05-E430G (right) after dosing days 1 (top) and 29 (bottom).

FIG. 7: Plasma concentration-time profiles following first IV dose of the mixture of IgG1-hDR5-01-G56T-E430G+IgG1-hDR5-05-E430G in human cancer patients. 15 patients in dose escalation cohorts were dosed: 0.3, 1.0 and 3.0 mg/kg. Graphs represent mean plasma concentration-time profiles for IgG1-hDR5-01-G56T-E430G and IgG1-hDR5-05-E430G.

FIG. 8: Cytotoxicity of HexaBody-DR5/DR5 in primary MM cells was analyzed ex vivo in 24-hour viability assays using BMNCs obtained from newly diagnosed (ND) and relapsed/refractory (RR) MM patients. (A) Median±interquartile range (IR) inhibition of cell viability of newly diagnosed (ND) versus relapsed and/or refractory (RR) MM by 20 μg/mL HexaBody-DR5/DR5. (B) Percentage MM cell kill by HexaBody-DR5/DR5 (20 μg/mL) of bone marrow (BM) samples from RR patients who had received their last exposure to therapy >1 month and month prior to their BM biopsy. *p<0.05 (Mann-Whitney test).

FIG. 9: Cytotoxicity of HexaBody-DR5/DR5 in combination with bortezomib in primary MM cells was analyzed ex vivo in 24-hour viability assays using bone marrow-derived mononuclear cells (BMNCs) obtained from (A) 11 newly diagnosed (ND) and (B) 6 relapsed/refractory (RR) MM patients. Median±IR inhibition of cell viability is presented. *p<0.05, **p<0.005 (Wilcoxon matched-pairs signed rank test).

FIG. 10: Cytotoxicity of HexaBody-DR5/DR5 in combination with lenalidomide in primary MM cells was analyzed ex vivo in 24-hour viability assays using BMNCs obtained from (A) 7 ND and (B) 6 RR MM patients. Median±IR inhibition of cell viability is presented. *p<0.05 (Wilcoxon matched-pairs signed rank test).

FIG. 11: Percentage inhibition of viability of lenalidomide-preincubated NCI-H929 cells by HexaBody-DR5/DR5, lenalidomide and the combination thereof. Cells were preincubated with 3 μM lenalidomide for 5 days before incubating for 24 h with HexaBody-DR5/DR5 and/or lenalidomide in concentrations below and above their EC50 values. Graphs show the mean and SD of duplicate measurements from a representative experiment (n=3).

FIG. 12: Inhibition of cell viability by HexaBody-DR5/DR5 in combination with lenalidomide on NCI-H929 cells in presence and absence of healthy donor peripheral blood mononuclear cells (PBMCs) in vitro. (A) Percentage kill of NCI-H929 cells by 20 μg/mL HexaBody-DR5/DR5 after a 24-hour incubation in the absence and presence of healthy donor PBMCs (n=6) in a 40:1 effector to target ratio. Mean and SD are shown. (ns) not significant (paired t-test). (B) Percentage inhibition of cell viability of NCI-H929 cells by 20 μg/mL HexaBody-DR5/DR5, 3 μM lenalidomide and the combination thereof in the presence of healthy donor PBMCs (n=7) that had been preincubated with 3 μM lenalidomide for 5 days. NCI-H929 cells were incubated with lenalidomide-exposed PBMCs and treated with lenalidomide and/or HexaBody-DR5/DR5 for an additional 24 h. Mean percentage kill and SD is shown. **p<0.005; ***p<0.0005 (paired t-test).

### DETAILED DISCLOSURE OF THE INVENTION

As described herein, the present invention relates to a combination of two DR5-specific antibodies (also referred to as “anti-DR5 ab” or “antibodies that bind DR5” herein) as defined in any aspect or embodiment herein, for use in combination with an immunomodulatory imide drug for the treatment of multiple myeloma.

### Definitions

The term “DR5”, as used herein, refers to death receptor 5, also known as CD262 and TRAILR2, which is a single-pass type I membrane protein with three extracellular cysteine-rich domains (CRDs), a transmembrane domain (TM) and a cytoplasmic domain containing a death domain (DD). In humans, the amino acid sequence encoding the DR5 protein shown in SEQ ID NO 55, is encoded by a nucleic acid sequence (UniProtKB-O-14763-1 TR10B_HUMAN).

The term “immunoglobulin” as used herein, refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain (HC) typically is comprised of a heavy chain variable region (abbreviated herein as VH) and a heavy chain constant region (CH). The heavy chain constant region of IgG antibodies typically is comprised of three domains, CH1, CH2, and CH3. The heavy chains are inter-connected via disulfide bonds in the so-called “hinge region”. Each light chain (LC) typically is comprised of a light chain variable region (abbreviated herein as VL) and a light chain constant region (CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)). Unless otherwise stated or contradicted by context, reference to human IgG1 amino acid positions in the present invention is according to the Eu-numbering (Edelman et al., Proc Natl Acad Sci U S A. 1969 May; 63(1):78-85; Kabat et al., Sequences of Proteins of Immunological Interest, Fifth Edition. 1991 NIH Publication No. 91-3242).

The term “hinge region” as used herein is intended to refer to the hinge region of an immunoglobulin heavy chain. Thus, for example the hinge region of a human IgG1 antibody corresponds to amino acids 216-230 according to the Eu numbering.

The term “CH2 region” or “CH2 domain” as used herein is intended to refer the CH2 region of an immunoglobulin heavy chain. Thus, for example the CH2 region of a human IgG1 antibody corresponds to amino acids 231-340 according to the Eu numbering. However, the CH2 region may also be any of the other isotypes or allotypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the Eu numbering. However, the CH3 region may also be any of the other isotypes or allotypes as described herein.

The term “fragment crystallizable region”, “Fc region”, “Fc fragment” or “Fc domain”, which may be used interchangeably herein, refers to an antibody region comprising, arranged from amino-terminus to carboxy-terminus, at least a hinge region, a CH2 domain and a CH3 domain. An Fc region of an IgG1 antibody can, for example, be generated by digestion of an IgG1 antibody with papain. The Fc region of an antibody may mediate the binding of the immunoglobulin to host tissues or factors, including various cells of the immune system (such as effector cells) and components of the complement system such as C1q, the first component in the classical pathway of complement activation.

The term “Fab fragment” in the context of the present invention, refers to a fragment of an immunoglobulin molecule, which comprises the variable regions of the heavy chain and light chain as well as the constant region of the light chain and the CH1 region of the heavy chain of an immunoglobulin. The “CH1 region” refers e.g. to the region of a human IgG1 antibody corresponding to amino acids 118-215 according to the Eu numbering. Thus, the Fab fragment comprises the binding region of an immunoglobulin.

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. An antibody may also be a multispecific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcΔAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody antibodies (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, WO2010129304A2), CrossMAbs (Roche, WO2011117329), LUZ-Y (Genentech), Biclonic (Merus, WO2013157953), Dual Targeting domain antibodies (GSK/Domantis), Two-in-one Antibodies or Dual action Fabs recognizing two targets (Genentech, Novlmmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (Medlmmune), IgG-like Bispecific (ImClone/Eli Lilly, Shen, J., et al. J Immunol Methods, 2007. 318(1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains ( [symbol] Bodies by Novlmmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (US007951918), SCORPIONS by Emergent BioSolutions/Trubion and Zymogenetics/BMS, Ts2Ab (Medlmmune/AZ (Dimasi, N., et al. J Mol Biol, 2009. 393(3): p. 672-92), scFv fusion by Genetech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb² by f-Star (WO2008/003116), and dual scFv-fusions, and like Fc fusions by HERA technology of Apogenix, nanobody-Fc fusions (such as from INHIBRX), MultYmab and MultYbody by JN Biosciences, Stradobody by Gliknik and Zybodies by Zyngenia. It also should be understood that the term antibody, unless specified otherwise, also includes polyclonal antibodies, monoclonal antibodies (such as human monoclonal antibodies), antibody mixtures (recombinant polyclonals) for instance generated by technologies exploited by Symphogen and Merus (Oligoclonics), multimeric Fc proteins as described in WO2015/158867, fusion proteins as described in WO2014/031646 and antibody-like polypeptides, such as chimeric antibodies and humanized antibodies. An antibody as generated can potentially possess any isotype.

The term “human antibody”, as used herein, refers to antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another species, such as a mouse, have been grafted onto human framework sequences.

The term “chimeric antibody”, as used herein, refers to an antibody in which both chain types i.e. heavy chain and light chain are chimeric as a result of antibody engineering. A chimeric chain is a chain that contains a foreign variable domain (originating from a non-human species, or synthetic or engineered from any species including human) linked to a constant region of human origin.

The term “humanized antibody, as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “isotype”, as used herein, refers to the immunoglobulin class (for instance IgG1, IgG2, IgG3, IgG4, IgD, IgA1, IgA2, IgE, or IgM) that is encoded by heavy chain constant region genes. To produce a canonical antibody, each heavy chain isotype is to be combined with either a kappa (κ) or lambda (λ) light chain.

The term “allotype”, as used herein, refers to the amino acid variation within one isotype class in the same species. The predominant allotype of an antibody isotype varies between ethnicity individuals. The known allotype variations within the IgG1 isotype of the heavy chain result from four amino acid substitutions in the antibody frame. In one embodiment the antibody of the invention is of the IgG1m(f) allotype as defined in SEQ ID NO 15. In one embodiment of the invention the first and second antibody of the invention is of the IgG1m(f) allotype as defined in SEQ ID NO 15, wherein at least one amino acid substitution has been introduced. In one embodiment of the invention the first and second antibody of the invention is of the IgG1m(f) allotype as defined in SEQ ID NO 15, wherein at most five amino acid substitutions has been introduced, such as four amino acid substitutions, such as three amino acid substitutions, such as two amino acid substitutions.

The terms “monoclonal antibody”, “monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity, which have variable and constant regions derived from human germline immunoglobulin sequences. The human mAbs may be generated by a hybridoma which includes a B cell obtained from a transgenic or transchromosomal non-human animal, such as a transgenic mouse, having a genome comprising a human heavy chain transgene repertoire and a human light chain transgene repertoire, rearranged to produce a functional human antibody and fused to an immortalized cell. Alternatively, the human mAbs may be generated recombinantly.

The term “full-length antibody” when used herein, refers to an antibody (e.g., a parent or variant antibody) which contains all heavy and light chain constant and variable domains corresponding to those that are normally found in a wild-type antibody of that class or isotype.

The term “oligomer” as used herein, refers to a molecule that consists of more than one but a limited number of monomer units (e.g. antibodies) in contrast to a polymer that, at least in principle, consists of an unlimited number of monomers. Exemplary oligomers are dimers, trimers, tetramers, pentamers and hexamers. Greek prefixes are often used to designate the number of monomer units in the oligomer, for example a tetramer being composed of four units and a hexamer of six units. Likewise, the term “oligomerization”, as used herein, is intended to refer to a process that converts molecules to a finite degree of polymerization. Herein, it is observed, that antibodies and/or other dimeric proteins comprising target-binding regions according to the invention can form oligomers, such as hexamers, via non-covalent association of Fc-regions after target binding, e.g., at a cell surface.

The term “antigen-binding region”, “antigen binding region”, “binding region” or antigen binding domain, as used herein, refers to a region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell, bacterium, or virion or in solution. The terms “antigen” and “target” may, unless contradicted by the context, be used interchangeably in the context of the present invention.

The term “target”, as used herein, refers to a molecule to which the antigen binding region of the antibody binds. The target includes any antigen towards which the raised antibody is directed. The term “antigen” and “target” may in relation to an antibody be used interchangeably and constitute the same meaning and purpose with respect to any aspect or embodiment of the present invention.

The term “epitope” means a protein determinant capable of specific binding to an antibody. Epitopes usually consist of surface groupings of building blocks such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked by the specifically antigen binding peptide (in other words, the amino acid residue is within the footprint of the specifically antigen binding peptide).

The term “binding” as used herein refers to the binding of an antibody to a predetermined antigen or target, typically with a binding affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less. Binding affinity may be determined by for instance surface plasmon resonance (SPR) technology in a BIAcore 3000 instrument using the antigen as the ligand and the antibody as the analyte or vice versa, and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low (that is, the antibody is highly specific), then the degree with which the affinity for the antigen is lower than the affinity for a non-specific antigen may be at least 10,000-fold. The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the koff value or off-rate.

The term “kₐ” (M⁻¹×sec⁻¹), as used herein, refers to the association rate constant of a particular antibody-antigen interaction. Said value is also referred to as the kₒₙ value or on-rate.

The term “K_{A}” (M⁻¹), as used herein, refers to the association equilibrium constant of a particular antibody-antigen interaction and is obtained by dividing kₐ by k_{d}.

As used herein, the term “affinity” is the strength of binding of one molecule, e.g. an antibody, to another, e.g. a target or antigen, at a single site, such as the monovalent binding of an individual antigen binding site of an antibody to an antigen.

As used herein, the term “avidity” refers to the combined strength of multiple binding sites between two structures, such as between multiple antigen binding sites of antibodies simultaneously interacting with a target. When more than one binding interactions are present, the two structures will only dissociate when all binding sites dissociate, and thus, the dissociation rate will be slower than for the individual binding sites, and thereby providing a greater effective total binding strength (avidity) compared to the strength of binding of the individual binding sites (affinity).

The term “hexamerization enhancing mutation”, as used herein, refers to a mutation of an amino acid position corresponding to E430, E345 or S440, with the proviso that the mutation in S440 is S440Y or S440W in human IgG1 according to Eu numbering. The hexamerization enhancing mutation strengthens Fc-Fc interactions between neighbouring IgG1 antibodies that are bound to a membrane target, resulting in enhanced hexamer formation of the target-bound antibodies, while the antibody molecules remain monomeric in solution as described in WO2013/004842; WO2014/108198.

The term “apoptosis”, as used herein refers to the process of programmed cell death (PCD) that may occur in a cell. Biochemical events lead to characteristic cell changes (morphology) and death. These changes include blebbing, cell shrinkage, phosphatidylserine exposure, loss of mitochondrial function, nuclear fragmentation, chromatin condensation, caspase activation, and chromosomal DNA fragmentation. In a particular embodiment, apoptosis by one or more agonistic anti-DR5 antibodies may be determined using caspase-3/7 activation assays or phosphatidylserine exposure. Anti-DR5 antibody at a fixed concentration of e.g. 1 μg/mL may be added to adhered cells and incubated for 1 to 24 hours. Caspase-3/7 activation can be determined by using special kits for this purpose, such as the PE Active Caspase-3 Apoptosis Kit of BD Pharmingen (Cat no 550914) or the Caspase-Glo 3/7 assay of Promega (Cat no G8091). Phosphatidylserine exposure and cell death can be determined by using special kits for this purpose, such as the FITC Annexin V Apoptosis Detection Kit I from BD Pharmingen (Cat no 556547).

The term “programmed cell death” or “PCD”, as used herein refers to the death of a cell in any form mediated by an intracellular signaling, e.g. apoptosis, autophagy or necroptosis.

The term “Annexin V”, as used herein, refers to a protein of the annexin group that binds phosphatidylserine (PS) on the cell surface.

The term “caspase activation”, as used herein, refers to cleavage of inactive pro-forms of effector caspases by initiator caspases, leading to their conversion into effector caspases, which in turn cleave protein substrates within the cell to trigger apoptosis.

The term “caspase-dependent programmed cell death”, as used herein refers to any form of programmed cell death mediated by caspases. In a particular embodiment, caspase-dependent programmed cell death by one or more anti-DR5 antibodies may be determined by comparing the viability of a cell culture in the presence and absence of pan-caspase inhibitor Z-Val-Ala-DL-Asp-fluoromethylketone (Z-VAD-FMK). Pan-caspase inhibitor Z-VAD-FMK (5 μM end concentration) may be added to cells in incubated for one hour at 37° C. Next, antibody concentration dilution series (e.g. starting from e.g. 20,000 ng/mL to 0.05 ng/mL final concentrations in 5-fold dilutions) may be added and incubated for 24 hours at 37° C. Cell viability can be quantified using special kits for this purpose, such as the CellTiter-Glo luminescent cell viability assay of Promega (Cat no G7571).

The term “cell viability”, as used herein refers to the presence of metabolically active cells in a cell culture. In a particular embodiment, cell viability after incubation with one or more anti-DR5 antibodies can be determined by quantifying the ATP present in the cells. Antibody concentration dilution series (e.g. starting from e.g. 20,000 ng/mL to 0.05 ng/mL final concentration in 5-fold dilutions) may be added to cells, medium may be used as negative control and 5 μM staurosporine may be used as positive control for the induction of cell death. After 24 hours incubation cell viability may be quantified using special kits for this purpose, such as the CellTiter-Glo luminescent cell viability assay of Promega (Cat no G7571) or ATPlite lstep Luminescence Assay System of Perkin Elmer (Cat no 6016739). The percentage viable cells can be calculated using the following formula: % viable cells=[(luminescence antibody sample−luminescence staurosporine sample)/(luminescence no antibody sample−luminescence staurosporine sample)]*100. Alternatively, the percentage of viable MM cells may be determined by flow cytometry and the live MM cell subset is identified as having the following profile (7AAD^{neg}/CD138^{pos}/CD38^{pos}). Percentage inhibition of viability (cell killing) in the MM cell populations may be calculated as follows: Viability inhibition=100%−[(viable MM cell counts in the test sample/average viable MM cell counts in the negative control samples)×100%].

The term “antibody capable of binding DR5”, “antibody binding DR5”, “anti-DR5 antibody”, “DR5-binding antibody”, “DR5-specific antibody”, “DR5 antibody” “antibody that binds DR5” or “antibodies that bind DR5” which may be used interchangeably herein, refers to any antibody binding an epitope on the extracellular part of DR5.”

The term “agonist” as used herein, refers to a molecule such as an anti-DR5 antibody that is able to trigger a response in a cell when bound to DR5, wherein the response may be programmed cell death.

That the anti-DR5 antibody is agonistic is to be understood as that the antibody stimulates, activates or clusters DR5 as the result from the anti-DR5 antibody binding to DR5. An agonistic anti-DR5 antibody comprising an amino acid mutation in the Fc region according to the present invention bound to DR5 results in DR5 stimulation, clustering or activation of the same intracellular signaling pathways as TRAIL bound to DR5.

In a particular embodiment, the agonist activity of one or more antibodies can be determined by incubating target cells for 24 hours with an antibody concentration dilution series (e.g. from 20,000 ng/mL to 0.05 ng/mL final concentrations in 5-fold dilutions). The antibodies may be added directly when cells are seeded, or alternatively the cells are first incubated for 4 h at 37° C. before adding the antibody samples. The agonist activity i.e. the agonist effect can be quantified by measuring the amount of viable cells using special kits for this purpose, such as the CellTiter-Glo luminescent cell viability assay of Promega (Cat no G7571) or ATPlite lstep Luminescence Assay System of Perkin Elmer (Cat no 6016739), or by flow cytometry.

The terms “DR5-positive” and “DR5-expressing” as used herein, refers to tissues or cells which show binding of a DR5-specific antibody which can be measured with e.g. flow cytometry or immunohistochemistry.

A “variant” or “antibody variant” of the present invention is an antibody molecule, which comprises one or more mutations as compared to a “parent” antibody. Exemplary parent antibody formats include, without limitation, a wild-type antibody, a full-length antibody or Fc-containing antibody fragment, a bispecific antibody, a human antibody, humanized antibody, chimeric antibody or any combination thereof.

The term “amino acid substitution” embraces a substitution into any one or the other nineteen natural amino acids, or into other amino acids, such as non-natural amino acids. For example, an amino acid may be substituted for another conservative or non-conservative amino acid. Amino acid residues may also be divided into classes defined by alternative physical and functional properties.

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln |
| | (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, and |
| | T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, |
| | S, P, and T |
| Flexible residues | Q, T, K, S, G, D, E, and R |

In the context of the present invention, a substitution in a variant is indicated as:

Original amino acid-position-substituted amino acid;

The three letter code, or one letter code, are used, including the codes Xaa and X to indicate amino acid residue. Accordingly, the notation “E345R” or “Glu345Arg” means, that the variant comprises a substitution of Glutamic acid with Arginine in the variant amino acid position corresponding to the amino acid in position 345 in the parent antibody.

Where a position as such is not present in an antibody, but the variant comprises an insertion of an amino acid, for example: Position—inserted amino acid; the notation, e.g., “448E” is used. Such notation is particular relevant in connection with modification(s) in a series of homologous polypeptides or antibodies.

For a modification where the original amino acid(s) and/or substituted amino acid(s) may comprise more than one, but not all amino acid(s), e.g., the substitution of Glutamic acid for Arginine, Lysine or Tryptophan in position 345: “Glu345Arg,Lys,Trp” or “E345R,K,W” or “E345R/K/W” or “E345 to R, K or W” may be used interchangeably in the context of the invention. Furthermore, the term “a substitution” embraces a substitution into any one of the other nineteen natural amino acids, or into other amino acids, such as non-natural amino acids. For example, a substitution of amino acid E in position 345 includes each of the following substitutions: 345A, 345C, 345D, 345G, 345H, 345F, 3451, 345K, 345L, 345M, 345N, 345Q, 345R, 345S, 345T, 345V, 345W, and 345Y. This is, by the way, equivalent to the designation 345X, wherein the X designates any amino acid. These substitutions can also be designated E345A, E345C, etc, or E345A,C, ect, or E345A/C/ect. The same applies to analogy to each and every position mentioned herein, to specifically include herein any one of such substitutions.

For the purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the

EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

For the purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et a/., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides x 100)/(Length of Alignment−Total Number of Gaps in Alignment).

The sequence of CDR variants may differ from the sequence of the CDR of the parent antibody sequences through mostly conservative, physical or functional amino acids substitutions at most 5 mutations or substitutions selected from conservative, physical or functional amino acids in total across the six CDR sequences of the antibody binding region, such as at most 4 mutations or substitutions selected from conservative, physical or functional amino acids, such as at most 3 mutations or substitutions selected from conservative, physical or functional amino acids, such as at most 2 mutations selected from conservative, physical or functional amino acids or substitutions, such as at most 1 mutation or substitution selected from a conservative, physical or functional amino acid, in total across the six CDR sequences of the antibody binding region. The conservative, physical or functional amino acids are selected from the 20 natural amino acids found i.e, Arg (R), His (H), Lys (K), Asp (D), Glu (E), Ser (S), Thr (T), Asn (N), Gln (Q), Cys (C), Gly (G), Pro (P), Ala (A), Ile (I), Leu (L), Met (M), Phe (F), Trp (W), Tyr (Y) and Val (V).

The sequence of CDR variants may differ from the sequence of the CDR of the parent antibody sequences through mostly conservative, physical or functional amino acids substitutions; for instance at least about 75%, about 80% or more, about 85% or more, about 90% or more, about 95% or more (e.g., about 75-99%, such as about 92%, 93% or 94%) of the substitutions in the variant are mutations or substitutions selected from conservative, physical or functional amino acids residue replacements. The conservative, physical or functional amino acids are selected from the 20 natural amino acids found i.e, Arg (R), His (H), Lys (K), Asp (D), Glu (E), Ser (S), Thr (T), Asn (N), Gln (Q), Cys (C), Gly (G), Pro (P), Ala (A), Ile (I), Leu (L), Met (M), Phe (F), Trp (W), Tyr (Y) and Val (V).

An amino acid or segment in one sequence that “corresponds to” an amino acid or segment in another sequence is one that aligns with the other amino acid or segment using a standard sequence alignment program such as ALIGN, ClustalW or similar, typically at default settings. Hence a standard sequence alignment program can be used to identify which amino acid in an e.g. immunoglobulin sequence corresponds to a specific amino acid in e.g. human IgG1. Further a standard sequence alignment program can be used to identify sequence identity e.g. a sequence identity to SEQ ID NO: 15 of at least 80%, or 85%, 90%, or at least 95%.

The term “vector,” as used herein, refers to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO-S cells, HEK-293F cells, Expi293F cells, PER.C6, NS0 cells, and lymphocytic cells, and prokaryotic cells such as E. coli and other eukaryotic hosts such as plant cells and fungi, as well as prokaryotic cells such as E. coli.

As used herein, a “derivative” of a drug is a compound that is derived or derivable, by a direct chemical reaction, from the drug. As used herein, an “analog” or “structural analog” of a drug is a compound having a similar structure and/or mechanism of action to the drug but differing in at least one structural element. “Therapeutically active” analogs or derivatives of a parent drug such may have a similar or improved therapeutic efficacy as compared to the parent drug but may differ in, e.g., one or more of stability, solubility, toxicity, and the like.

“Treatment” refers to the administration of an effective amount of a therapeutically active compound as described herein to a subject with the purpose of easing, ameliorating, arresting or eradicating (curing) symptoms or disease states of the subject.

As used herein, “maintenance therapy” means therapy for the purpose of avoiding or delaying the cancer's progression or return. Typically, if a cancer is in complete remission after the initial treatment, maintenance therapy can be used to avoid or delay return of the cancer. If the cancer is advanced and complete remission has not been achieved after the initial treatment, maintenance therapy can be used to slow the growth of the cancer, e.g., to lengthen the life of the patient.

As used herein, the term “subject” is typically a human, to whom a first and second antibody binding to DR5 is administered, including for instance human patients diagnosed as having a cancer that may be treated by killing of DR5-expressing cancer cells, directly or indirectly.

An “effective amount” or “therapeutically effective amount” refers to an amount effective, at dosages and for periods of time necessary, to achieve a desired therapeutic result. A therapeutically effective amount of a first and second anti-DR5 antibody may vary according to factors such as the disease state, age, sex, and weight of the individual, and the ability of the first and second anti-DR5 antibody to elicit a desired response in the individual. A therapeutically effective amount is also one in which any toxic or detrimental effects of the first and second anti-DR5 are outweighed by the therapeutically beneficial effects.

The term a “cycle” or “cycle of treatment” describes a period of treatment followed by a period of rest (no treatment) that is repeated on a regular schedule. For example, treatment given on day 1 followed by 13 days of rest is one treatment cycle of 14-days. When this cycle is repeated multiple times on a regular schedule, it makes up a course of treatment. In one embodiment the treatment is administered on day 1 of a 14 days cycle. In one embodiment the treatment is administered on day 1 and day 8 of a 14-days cycle. Alternatively, a treatment cycle may also be defined as 7-days, so that the treatment is e.g. administered on day one of a 7-days cycle i.e. treatment is administered on day 1 followed by 6 days of rest, one treatment cycle is 7-days. For immunomodulatory imide drugs such as lenalidomide the cycle of treatment may be defined as a cycle of 28-days. Thus, immunomodulatory imide drugs, such as lenalidomide may be administered daily on day 1 to day 21 of a 28-days treatment cycle. Thus, immunomodulatory imide drugs, such as lenalidomide may be administered daily for 21 days followed by 7 days of rest amounting to a 28-day cycle. The treatment period for a first and a second antibody may also be described according to a cycle of 28-days, thus in on embodiment of the invention the first and second antibody may be administered on day 1 and day 15 of a 28-days treatment cycle. In another embodiment of the invention the first and second antibody may be administered on day 1, 8, 15 and 22 of a 28-days treatment cycle.

The term “Ctrough” describes the drug serum concentration at the end of the dosing interval. Thus, Ctrough is the lowest concentration reached by a drug before the next dose is administered.

The term “Therapeutic Index” (TI) describes the ratio of the dose of drug that causes adverse effects at an incidence/severity not compatible with the targeted indication (e.g. toxic dose in 50% of subjects, TD50) to the dose that leads to the desired pharmacological effect (e.g. efficacious dose in 50% of subjects, ED50).

As used herein, a “resistant”, “treatment-resistant” cancer, tumor or the like, means a cancer or tumor in a subject, wherein the cancer or tumor did not respond to treatment with a therapeutic agent from the onset of the treatment (herein referred to as “native resistance”) or initially responded to treatment with the therapeutic agent but became non-responsive or less responsive to the therapeutic agent after a certain period of treatment (herein referred to as “acquired resistance”), resulting in progressive disease. For solid tumors, also an initial stabilization of disease represents an initial response. Other indicators of resistance include recurrence of a cancer, increase of tumor burden, newly identified metastases or the like, despite treatment with the therapeutic agent. Whether a tumor or cancer is, or has a high tendency of becoming resistant to a therapeutic agent, can be determined by a person of skill in the art. For example, the National Comprehensive Cancer Network (NCCN, www.nccn.org) and European Society for Medical Oncology (ESMO, www.esmo.org/Guidelines) provide guidelines for assessing whether a specific cancer responds to treatment.

The term “Multiple myeloma” or “MM” as used herein describes a hematologic malignancy characterized by clonal proliferation of abnormal plasma cells in the bone marrow.

The term “refractory” or “refractory multiple myeloma” as used herein describes a disease, such as multiple myeloma that is nonresponsive to treatment in patients who have never achieved a minimal response or better with any therapy. It includes MM patients who never achieve minimal response or better with any therapy in whom there is no significant change in M protein and no evidence of clinical progression as well as primary refractory, progressive disease (PD) where patients meet criteria for true PD. Rajkumar, S. V., et al., Blood, 2011. 117(18): p. 4691-5.

The term “relapsed-and-refractory” or “relapsed and refractory multiple myeloma” as used herein describes a disease, such as multiple myeloma that is nonresponsive while on salvage therapy or progresses within 60 days of last therapy in patients who have achieved minimal response or better at some point previously before then progressing in their disease course. Rajkumar, S. V., et al., Consensus recommendations for the uniform reporting of clinical trials: report of the International Myeloma Workshop Consensus Panel 1. Blood, 2011. 117(18): p. 4691-5.

The term “relapsed” or “relapsed multiple myeloma” as used herein describes a previously treated disease, such as multiple myeloma that progresses and requires the initiation of salvage therapy but does not meet criteria for either “refractory multiple myeloma” or “relapsed-and-refractory multiple myeloma” categories. Rajkumar, S. V., et al., Blood, 2011. 117(18): p. 4691-5.

The term “relapsed and/or refractory”, or “RR” or “RR multiple myeloma” as used herein describes a disease, such as multiple myeloma that is either relapsed, refractory or relapsed and refractory.

The term “Immunomodulatory drug” or “Immunomodulatory drugs” as used herein describes a class of drugs that modify the immune responses.

The term “Immunomodulatory imide drug” “Immunomodulatory imide drugs” or “IMiDs” are a class of drugs that modify the immune responses containing an imide group. The IMiD class includes thalidomide and its analogues, i.e. lenalidomide, pomalidomide, iberdomide, and apremilast.

### Specific Embodiments of the Invention

As explained above, the invention is directed to a combination treatment for multiple myeloma involving a first antibody capable of binding to DR5 and a second antibody capable of binding to DR5, wherein the treatment has been improved by further combining the antibodies with an immunomodulatory imide drug.

In one aspect, the present invention relates to a method of treating multiple myeloma in a subject, the method comprising administering to a subject in need thereof a first antibody capable of binding DR5 and a second antibody capable of binding DR5, or a pharmaceutically acceptable salt thereof, in combination with an immunomodulatory imide drug. Thus, the present invention may provide for an improved method of treating multiple myeloma by enhancing the effect of the first and second anti-DR5 antibodies and the immunomodulatory imide drug, compared to when they are used alone i.e., either the first and second anti-DR5 antibodies or the immunomodulatory imide drug.

Preferred anti-DR5 antibodies are characterized by DR5 binding properties, variable or hypervariable sequences, or a combination of binding and sequence properties, set out in the aspects and embodiments below. Most preferred are the specific anti-DR5 antibodies comprising VH region and VL region CDRs, VH and/or VL sequences described in Table 2 of particular interest are antibodies sharing one or more DR5 binding properties or CDRs, VH and/or VL sequences with an antibody seleed from the roup consisting of antibody hDR5-01 and antibody hDR5-05 and or a variant of any thereof.

In one embodiment, the antibody capable of binding to DR5 comprises a variable heavy chain (VH) region and a variable light chain (VL) region, wherein the VH region and VL region comprises the CDR sequences selected from the group consisting of
(a) a VH region comprising the CDR1, CDR2, and CDR3 sequences of SEQ ID Nos.: 1, 2, and 3, respectively; and a VL region comprising the CDR1, CDR2, and CDR3 sequences of SEQ ID Nos.: 5, FAS, and 6, respectively, [hDR5-01-G56T];
(b) a VH region comprising the CDR1, CDR2, and CDR3 sequences of SEQ ID Nos.: 8, 9, and 10, respectively, and a VL region comprising the CDR1, CDR2, and CDR3 sequences of SEQ ID Nos.: 12, RTS, and 13, respectively [hDR5-05];
(c) a variant of any of said antibodies defined in (a) to (b), wherein said variant preferably has at most 1, 2 or 3 amino acid modifications, more preferably amino acid substitutions, such as conservative amino acid substitutions, across the six CDR sequences. Thus, in one embodiment the first and the second antibody may be selected from an antibody as described in (a) and (b) where the first and second antibody is not the same.

In one embodiment, the first or second antibody capable of binding DR5 comprises a variable heavy chain (VH) region and a variable light chain (VL) region wherein the variable heavy chain region comprises the CDR1, CDR2 and CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

In one embodiment, the first or second antibody capable of binding DR5 comprises a variable heavy chain (VH) region and a variable light chain (VL) region region wherein the variable heavy chain region comprises the CDR1, CDR2 and CDR3 sequences of SEQ ID Nos: 8, 9, and 10 respectively; and wherein the variable light chain region comprises the CDR1, CDR2 and CDR3 sequences of SEQ ID Nos: 12, RTS, and 13, respectively.

In one embodiment, the first antibody capable of binding DR5 comprises a variable heavy chain region and a variable light chain region wherein the variable heavy chain region comprises the CDR1, CDR2 and

CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

In one embodiment, the second antibody capable of binding DR5 comprises a variable heavy chain region and a variable light chain region wherein the variable heavy chain region comprises the CDR1, CDR2 and CDR3 sequences of SEQ ID Nos: 8, 9, and 10 respectively; and wherein the variable light chain region comprises the CDR1, CDR2 and CDR3 sequences of SEQ ID Nos: 12, RTS, and 13, respectively.

In one embodiment of the invention, the first or second antibody capable of binding DR5 comprises a VH region and a VL region selected from the group consisting of:
(a) a VH region comprising SEQ ID No: 4 and a VL region comprising SEQ ID No: 7 [hDR5-01-G56T]; and
(b) a VH region comprising SEQ ID No: 11 and a VL region comprising SEQ ID No: 14 [hDR5-05].

In one preferred embodiment of the invention, the first antibody capable of binding DR5 is an antibody having the VH region CDR1, CDR2 and CDR3 amino acid sequences set forth in SEQ ID Nos: 1, 2, and 3, respectively; and the VL region CDR1, CDR2 and CDR3 amino acid sequence set forth in SEQ ID Nos: 5, FAS, and 6, respectively, [hDR5-01-G56T] and the second antibody capable of binding DR5 is an antibody having the VH region CDR1, CDR2 and CDR3 amino acid sequences set forth in SEQ ID Nos: 8, 9, and 10, respectively; and the VL region CDR1, CDR2 and CDR3 amino acid sequence set forth in SEQ ID Nos: 12, RTS, and 13, respectively, [hDR5-05].

For example, the first antibody capable of binding DR5 may comprise a VH region comprising SEQ ID No: 4 and a VL region comprising SEQ ID No: 7 [hDR5-01-G56T]; and the second antibody capable of binding DR5 may comprise a VH region comprising SEQ ID No: 11 and a VL region comprising SEQ ID No: 14 [hDR5-05].

In one embodiment of the invention, the first and second antibody bind different epitopes on DR5. Hereby are embodiments provided where the antibodies bind different epitopes or require different amino acids within the DR5 sequence (SEQ ID No: 41) for binding to DR5. In one embodiment of the invention the first and second antibody bind non-overlapping epitopes on DR5. That is in one embodiment of the invention the first and second antibodies binding to DR5 do not compete for binding to DR5, thus the first and second antibody may bind DR5 simultaneously.

In a preferred embodiment of the invention, the antibody is a full-length antibody. The antibody may, for example, be a fully human monoclonal IgG1 antibody, such as an IgG1,κ. In one embodiment, the antibody is a full-length antibody.

In one embodiment of the invention, the antibody capable of binding to DR5 comprises an Fc region of a human IgG1, wherein the Fc region comprises a mutation which enhances Fc-Fc interactions between antibodies. Mutations which have been shown to enhance Fc-Fc interactions are mutations at an amino acid position corresponding to E430, E345, or S440 in human IgG1 according to Eu numbering, with the proviso that the mutation in S440 is S440Y or S440W. Mutations that enhance Fc-Fc interactions has also been found to enhance hexamerization of antibodies comprising such Fc-Fc enhancing mutations, once such antibodies bind to their target on a cell membrane surface.

In one embodiment of the invention, the antibody capable of binding to DR5 comprises an Fc region of human IgG1, wherein the Fc region comprises a mutation at the amino acid position corresponding to E430. In one embodiment the antibody binding to DR5 comprises an Fc region of human IgG1, wherein the Fc region comprises a mutation at the amino acid position corresponding to E345. In one embodiment the antibody binding to DR5 comprises an Fc region of human IgG1, wherein the Fc region comprises a S440Y or S440W mutation.

In one embodiment of the invention, the first and/or second antibody comprises a mutation at the amino acid position corresponding to E430 in human IgG1 according to Eu numbering, wherein the mutation is selected from the group consisting of: E430G, E430S, E430F and E430T.

In one embodiment of the invention, the first and/or second antibody comprises a mutation at the amino acid position corresponding to E345 in human IgG1 according to Eu numbering, wherein the mutation is selected form the group consisting of: E345K, E345Q, E345R and E345Y.

In one embodiment of the invention, the first and/or second antibody comprises a mutation corresponding to S440Y or S440W in human IgG1 according to Eu numbering.

In one embodiment of the invention, the first and second antibody comprises an Fc region of a human IgG1, wherein the Fc region comprises an E430G mutation of an amino acid position corresponding E430 in human IgG1, wherein the amino acid position is according to the Eu numbering.

In one embodiment of the invention, the first or second antibody comprises the heavy chain set forth in SEQ ID NO 17. In one embodiment of the invention the first or second antibody comprises the heavy chain set forth in SEQ ID NO 19.

In one embodiment of the invention, the first or second antibody comprises the heavy chain set forth in SEQ ID NO 18. In one embodiment of the invention the first or second antibody comprises the heavy chain set forth in SEQ ID NO 19.

In one embodiment of the invention, the first or second antibody comprises the light chain set forth in SEQ ID NO 21. In one embodiment of the invention the first or second antibody comprises the light chain set forth in SEQ ID NO 22.

In one embodiment of the invention, the first or second antibody comprises the heavy chain and light chain as set forth in SEQ ID Nos 20 and 22, respectively.

In one embodiment of the invention, the first antibody comprises the heavy chain and light chain as set forth in SEQ ID Nos 17 and 19, respectively. In one embodiment of the invention the second antibody comprises the heavy chain and light chain as set forth in SEQ ID NOs 20 and 22, respectively.

In one embodiment of the invention, the first antibody comprises the heavy chain and light chain as set forth in SEQ ID NOs 18 and 19, respectively. Hereby an embodiment is provided where the C-terminal lysine has been removed from the heavy chain, thus allowing for a more homogeneous antibody.

In one embodiment of the invention, the second antibody comprises the heavy chain and light chain as set forth in SEQ ID NOs 21 and 22, respectively. Hereby an embodiment is provided where the C-terminal lysine has been removed from the heavy chain, thus allowing for a more homogeneous antibody.

In one embodiment, the immunomodulatory imide drug is thalidomide or a thalidomide analog, e.g.

lenalidomide or pomalidomide. Hereby embodiments are provided which may allow for an enhanced therapeutic effect of an antibody capable of binding to DR5 when said anti-DR5 antibody is used in treatment of multiple myeloma.

In one embodiment, the immunomodulatory imide drug is selected from the group consisting of thalidomide, lenalidomide, pomalidomide and apremilast. In one embodiment of the invention, the immunomodulatory imide drug is thalidomide. In one embodiment of the invention, the immunomodulatory imide drug is pomalidomide. In one embodiment of the invention, the immunomodulatory imide drug is apremilast. In a preferred embodiment of the invention, the immunomodulatory imide drug is lenalidomide.

### Therapeutic Applications

The present invention provides for methods of treating multiple myeloma in a subject by administering a first and a second antibody capable of binding to DR5 and an immunomodulatory imide drug as described herein.

The present invention includes embodiments wherein a subject suffers from relapsed and/or multiple myeloma. In one embodiment the multiple myeloma is relapsed multiple myeloma. In one embodiment, the multiple myeloma is refractory multiple myeloma. In one embodiment the multiple myeloma is relapsed and/or refractory multiple myeloma.

The subject to be treated according to the present invention is typically a subject expected to benefit from the administration of a first and a second capable of binding to DR5. In separate and specific exemplary embodiments, the subject to be treated according to the present invention is selected from:
a subject that has been diagnosed with multiple myeloma,
a subject that has been diagnosed with relapsed multiple myeloma,
a subject that has been diagnosed with refractory multiple myeloma,
a subject that has been diagnosed with relapsed and refractory multiple myeloma
a subject diagnosed with a multiple myeloma which is resistant, or which has a high tendency to become resistant, to certain therapeutic agent(s).

Thus, a subject who may benefit from a treatment according to the present invention may have been treated with a therapeutic agent selected form the following category of drugs: a proteasome inhibitor, an immunosuppressor, an antibody, an anti-angiogenic, cytostatic agents and immunomodulator.

More specifically a subject who may benefit from the treatment according to the present invention may previously have been treated with one or more of the following therapeutic agents: bortezomib (Velcade), carfilzomib (Kyprolis), dexamethasone, prednisone, cyclophosphamide, thalidomide, pomalidomide, lenalidomide (Revlimid), bendamustine (Treanda), melphalan, doxorubicin (Adriamycine), daratumumab (Darzalex), durvalumab (Imfinzi), isatuximab, stem cell transplantation, donor lymphocyte infusion).

In one embodiment of the invention, the first and second antibody, or a pharmaceutically acceptable salt thereof, are administered simultaneously, separately, or sequentially. In one embodiment of the invention the first and second antibody, or a pharmaceutically acceptable salt thereof, are administered simultaneously. That is, the first and second antibody may be stored separately, but mixed together to a single solution before administration, so that the first and second antibody may be administered simultaneously. In one embodiment of the invention the first and second antibody, or pharmaceutically acceptable salt thereof, are administered separately. In one embodiment of the invention the first and second antibody, or a pharmaceutically acceptable salt thereof, are administered sequentially. That is, the first antibody may be administered to the subject first followed by administration of the second antibody. Alternatively, the second antibody may be administered to the subject first followed by administration of the first antibody.

In one embodiment of the invention, the first or second antibody, or a pharmaceutically acceptable salt thereof, is administered by intravenous infusion.

In one embodiment of the invention, the first and second antibody, or a pharmaceutically acceptable salt thereof, are administered by intravenous infusion.

The present invention includes embodiments wherein the a first and a second antibody capable of binding DR5 or a pharmaceutically acceptable salt thereof is/are administered on i) day 1 of a 7-days cycle or ii) day 1 of a 14-days cycle (1Q2W).

In one embodiment of the invention, a first and a second antibody capable of binding DR5 or a pharmaceutically acceptable salt thereof are administered on day 1 of a 7-days cycle.

In one embodiment of the invention, a first or a second antibody capable of binding DR5 or a pharmaceutically acceptable salt thereof is administered on day 1 of a 7-days cycle.

In one embodiment of the invention, a first and a second antibody capable of binding DR5 or a pharmaceutically acceptable salt thereof are administered on day 1 of a 14-days cycle.

In one embodiment of the invention, a first or a second antibody capable of binding DR5 or a pharmaceutically acceptable salt thereof is administered on day 1 of a 14-days cycle.

In one embodiment of the invention, a first and a second antibody capable of binding DR5 or a pharmaceutically acceptable salt thereof are administered on day 1, 8, 15 and 22 of a 28-days cycle.

In one embodiment of the invention, a first or a second antibody capable of binding DR5 or a pharmaceutically acceptable salt thereof is administered on day 1, 8, 15 and 22 of a 28-days cycle.

In one embodiment of the invention, a first and a second antibody capable of binding DR5 or a pharmaceutically acceptable salt thereof are administered on day 1 and 15 of a 28-days cycle.

In one embodiment of the invention, a first or a second antibody capable of binding DR5 or a pharmaceutically acceptable salt thereof is administered on day 1 and 15 of a 28-days cycle.

In some embodiments, the first doses administered is a priming dose which is a reduced dose compared to the following doses administered to the subject. Thus, the priming dose may allow for desensitization of the subjects to potential toxicities of higher doses. The effect of administering a priming dose may mitigate potential transaminase elevations caused by administration of the first and second antibody binding to DR5. Thus, administering a priming dose may reduce, prevent or lessen the induction of transaminase levels by the first and second antibody, such as reduce, prevent or lessen the induction of alanine transaminase (ALT) or aspartate transaminase (AST). In one embodiment the priming dose of the first and/or second antibody or a pharmaceutically acceptable salt thereof, is/are administered at a dose ranging from about 0.05 mg/kg to 0.3 mg/kg. In one embodiment, the priming dose of the first or second antibody or a pharmaceutically acceptable salt thereof, is administered at a dose ranging from about 0.05 mg/kg to 0.3 mg/kg. In one embodiment the priming dose of the first and second antibody or a pharmaceutically acceptable salt thereof, are administered at a dose ranging from about 0.05 mg/kg to 0.3 mg/kg. In one embodiment the combined priming dose of the first and second antibody is in the range of 0.1 mg/kg to 0.3 mg/kg. In a preferred embodiment the combined priming dose of the first and second antibody is 0.1 mg/kg.

In one embodiment, the first and/or second antibody, or a pharmaceutically acceptable salt thereof, is/are administered at a dose ranging from about 0.05 mg/kg to 18 mg/kg, such as from 0.05 mg/kg to 6 mg/kg.

The treatment dose administered following the priming dose is in the range of 0.15 mg/kg to 18 mg/kg for each first and second antibody. In one embodiment following the first or first and second priming dose the subject is administered as a treatment dose on a schedule of one dose every two weeks, wherein the treatment dose is in the range of 0.15 mg/kg to 9 mg/kg for each first and second antibody. In one embodiment following the first or first and second priming dose, the subject is administered a treatment dose on a schedule of one dose every two weeks, wherein the treatment dose is in the range of 0.3 mg/kg to 18 mg/kg for the combined dose of the first and second antibody. In a preferred embodiment the treatment dose of the first and second antibody combined is in the range of 0.3 mg/kg to 6 mg/kg. In a more preferred embodiment, the treatment dose of the first and second antibody combined is in the range of 0.3 mg/kg to 3 mg/kg.

In one embodiment, the first and/or second antibody, or a pharmaceutically acceptable salt thereof, is/are administered at a dose of about 0.05 mg/kg, 0.1 mg/kg, 0.15 mg/kg, 0.3 mg/kg, 0.5 mg/kg, 0.6 mg/kg, 1 mg/kg, 1.5 mg/kg, 2.0 mg/kg , 2.25 mg/kg, 3 mg/kg, 4.5 mg/kg, 6 mg/kg, 7.5 mg/kg, 9 mg/kg, 12 mg/kg, 15 mg/kg, 18 mg/kg.

### Protocol

In one aspect, the present invention provides for methods of treating a subject with multiple myeloma as described herein wherein the first and second antibody or pharmaceutically acceptable salt thereof are administered at a particular frequency.

The present invention includes embodiments wherein a subject will be administered a first and a second antibody capable of binding DR5, where the first and second antibody or a pharmaceutically acceptable salt thereof are administered on day 1 of a first 14-days cycle (priming); followed by administration on day 1 of a 14-days cycle (1Q2W). Thus, following the initial dosage schedule according to which may allow for desensitization of the subjects to the therapy and reduce potential toxicities of higher doses of treatment the subject may receive treatment with a higher dose administered based once every two weeks.

In a preferred embodiment, the first and second antibody is administered as a single priming dose on day 1 of a 14-day cycle, followed by administration of a treatment dose on day 1 of a 14-day cycle. In a preferred embodiment of the invention, the priming dose is 0.05 mg/kg of each of the first and second antibody. In a preferred embodiment of the invention, the priming dose is 0.1 mg/kg of the first and second antibody combined.

In one embodiment of the invention, the first and/or second antibody, or a pharmaceutically acceptable salt thereof, is/are administered to the subject on day 1 of a first 14-day cycle at a dose ranging from about 0.05 mg/kg to 1 mg/kg, such as ranging from about 0.05 mg/kg to 0.3 mg/kg.

In one embodiment of the invention, the first and/or second antibody, or a pharmaceutically acceptable salt thereof, is/are administered to the subject on day 1 of a first and second 14-day cycle at a dose ranging from about 0.05 mg/kg to 1 mg/kg, such as ranging from about 0.05 mg/kg to 0.3 mg/kg.

In one embodiment of the invention, where the first and second antibody or a pharmaceutically acceptable salt thereof are combined, the total amount of antibody administered is at a dose ranging from about 0.1 mg/kg to 18 mg/kg.

In one embodiment of the invention, the treatment dose administered following the priming dose is within the range of 0.1 mg/kg to 18 mg/kg for the combined dose of the first and second antibody. In a preferred embodiment of the invention, the treatment dose administered following the priming dose is within the range of 0.3 mg/kg to 6 mg/kg for the combined dose of the first and second antibody. In one embodiment of the invention, the treatment dose of the first and second antibody is 0.3 mg/kg. In one embodiment of the invention, the treatment dose of the first and second antibody is 0.6 mg/kg. In one embodiment of the invention, the treatment dose of the first and second antibody is 1 mg/kg. In one embodiment of the invention, the treatment dose of the first and second antibody is 2 mg/kg. In one embodiment of the invention, the treatment dose of the first and second antibody is 3 mg/kg. In one embodiment of the invention, the treatment dose of the first and second antibody is 4 mg/kg. In one embodiment of the invention, the treatment dose of the first and second antibody is 4.5 mg/kg. In one embodiment of the invention, the treatment dose of the first and second antibody is 6 mg/kg. In one embodiment of the invention, the treatment dose of the first and second antibody is 9 mg/kg. In one embodiment of the invention, the treatment dose of the first and second antibody is 12 mg/kg. In one embodiment of the invention, the treatment dose of the first and second antibody is 15 mg/kg. In one embodiment of the invention, the treatment dose of the first and second antibody is 18 mg/kg. Hereby embodiments are provided wherein the treatment dose is presented as the combined dose of the first and second antibody.

In one embodiment of the present invention, the first and second antibody binding to DR5, or pharmaceutical acceptable salt thereof, are administered once in a first 14-days cycle as a priming dose followed by continued administration on day 1 of a 14-days cycle (1Q2W). In one embodiment of the invention the first and second antibody binding to DR5, or pharmaceutical acceptable salt thereof, are administered on day 1, 2 or 3 of the first 14-days cycles (priming), followed by continued administration on day 1, 2 or 3 of a 14-day cycle. In one embodiment of the invention the first and second antibody binding to DR5, or pharmaceutical acceptable salt thereof, are administered on day 1 of the first 14-day cycle (priming), followed by administration on day 1 of a 14-day cycle (1Q2W). Thus, the administration of the first and second antibody binding to DR5 in the first 14-days cycles is the administration according to a priming regimen, which allows for desensitization of the subjects to the therapy and reduce potential toxicities of higher doses of treatment. Thus, following the initial priming doses the subject may receive treatment administered based on a biweekly dosage regimen, where the following doses are a higher dose than the priming doses. The priming dose administered is a lower dose of the first and second antibody binding to DR5 than the dose administered in the following 14-day cycles. Thus, the first priming dose may be of 0.1 mg/kg whereas the following doses may be from 0.3 mg/kg to 18 mg/kg. Thus, the priming dose may be a lower dose than the following doses administered to the subject. The priming doses used at the initiation of therapy may be used for desensitization of the subjects to the therapy and thereby the priming dose(s) may reduce potential toxicities of higher doses of treatment.

In one embodiment of the present invention, the first and second antibody binding to DR5, or pharmaceutical acceptable salt thereof, are administered once in a first and second 14-days cycle as a priming dose followed by continued administration on day 1 of a 14-days cycle (1Q2W). In one embodiment of the invention the first and second antibody binding to DR5, or pharmaceutical acceptable salt thereof, are administered on day 1, 2 or 3 of the first and second 14-days cycles (priming), followed by continued administration on day 1, 2 or 3 of a 14-day cycle. In one embodiment of the invention the first and second antibody binding to DR5, or pharmaceutical acceptable salt thereof, are administered on day 1 of the first and second 14-days cycles (priming), followed by administration on day 1 of a 14-day cycle (1Q2W). Thus, the administration of the first and second antibody binding to DR5 in the first and second 14-days cycles is the administration according to a priming regimen, which allow for desensitization of the subjects to the therapy and reduce potential toxicities of higher doses of treatment. Thus, following the initial priming doses the subject may receive treatment administered based on a biweekly dosage regimen, where the following doses are a higher dose than the priming doses. The priming dose administered is a lower dose of the first and second antibody binding to DR5 than the dose administered in the following 14-day cycles. Thus, the first priming dose may be of 1 mg/kg and the second priming dose may be from 1 mg/kg to 6 mg/kg, whereas the following doses may be from 3 mg/kg to 15 mg/kg. Thus, the priming dose may be a lower dose than the following doses administered to the subject. The priming doses used at the initiation of therapy may be used for desensitization of the subjects to the therapy and thereby the priming dose(s) may reduce potential toxicities of higher doses of treatment.

The present invention encompasses embodiments wherein the subject remains on the biweekly (1Q2W) treatment cycle, such as on day 1 of a 14-days cycle for at least 2, 3, 4, 5, 6, 7, 8, 9, 10, 11, 12 or more cycles. In another embodiment, the subject remains on the biweekly treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles wherein each cycle is 14 days as described above. In some embodiments, the subject remains on the 1Q2W treatment cycle for 12 cycles or more, such as 16 cycles or more, such as 24 cycles or more, such as 36 cycles or more. In some embodiments, the first and second antibodies are administered for no more than 3, no more than 4, no more than 5, or no more than 6, no more than 7, no more than 8, no more than 9, no more than 10, no more than 11, no more than 12 14-days treatment cycles. The number of treatment cycles suitable for any specific subject or group of subjects may be determined by a person of skill in the art, typically a physician. For example, such a person may evaluate the response to the anti-DR5 antibody treatment based on the criteria provided in Table 1, IMWG criteria.

In certain embodiments of the invention, the first or second antibody, or a pharmaceutically acceptable salt thereof, is administered at a dose ranging from about 0.05 mg/kg to 9 mg/kg or about 0.15 mg/kg to 18 mg/kg. Thus, the dosage may be adjusted to the subject's body weight. In one embodiment of the invention, the first or second antibody, or a pharmaceutically acceptable salt thereof, is administered at a dose ranging from about 0.05 mg/kg to 6 mg/kg or about 0.15 mg/kg to 9 mg/kg. In one embodiment of the invention, the first or second antibody, or a pharmaceutically acceptable salt thereof, is administered at a dose of about 0.05 mg/kg, or a dose of about 0.15 mg/kg, or a dose of about 0.3 mg/kg, or a dose of about 0.5 mg/kg, or a dose of about 1 mg/kg, or a dose of about 1.5 mg/kg, or a dose of about 2.25 mg/kg, or a dose of about 3 mg/kg, or a dose of about 4.5 mg/kg, or a dose of about 6 mg/kg, or a dose of about 7.5 mg/kg, or a dose of about 9 mg/kg. In one embodiment of the invention, the first or second antibody, or a pharmaceutically acceptable salt thereof, is administered at dose range of about 0.1 mg/kg to 3 mg/kg or about 1 mg/kg to 6 mg/kg. In one embodiment of the invention, the first or second antibody, or a pharmaceutically acceptable salt thereof, is administered at a dose of about 0.05 mg/kg, 0.15 mg/kg, 0.3 mg/kg, 0.5 mg/kg, 1 mg/kg, 1.5 mg/kg, 2.25 mg/kg, 3 mg/kg, 4.5 mg/kg, 6 mg/kg, 7.5 mg/kg or 9 mg/kg.

In one embodiment of the invention, the first or second antibody, or a pharmaceutically acceptable salt thereof, is administered to the subject on day 1 of a first 14-days cycle at a dose ranging from about 0.05 mg/kg to 0.15 mg/kg. In one embodiment of the invention, the first or second antibody, or a pharmaceutically acceptable salt thereof, is administered to the subject on day 1 of a first 14-days cycle at a dose of 0.05 mg/kg. In one embodiment of the invention, the first or second antibody, or a pharmaceutically acceptable salt thereof, is administered to the subject on day 1 of a first 14-days cycle at a dose of 0.15 mg/kg. In one embodiment of the invention, the first or second antibody, or a pharmaceutically acceptable salt thereof, is administered to the subject on day 1 of a first 14-days cycle at a dose of 0.30 mg/kg. In one embodiment of the invention, the first or second antibody, or a pharmaceutically acceptable salt thereof, is administered to the subject on day 1 of a first 14-days cycle at a dose of 0.5 mg/kg. In one embodiment of the invention, the first or second antibody, or a pharmaceutically acceptable salt thereof, is administered to the subject on day 1 of a first 14-days cycle at a dose of 1 mg/kg. In one embodiment of the invention, the first or second antibody, or a pharmaceutically acceptable salt thereof, is administered to the subject on day 1 of a first 14-days cycle at a dose of 2 mg/kg.

In one embodiment of the invention, the first or second antibody, or a pharmaceutically acceptable salt thereof is administered to the subject on day 1 of a first 14-day cycle at a dose ranging from about 0.05 mg/kg to 1 mg/kg, such as ranging from about 0.05 mg/kg to 0.3 mg/kg.

In one embodiment of the invention, the first and second antibody, or a pharmaceutically acceptable salt thereof, are combined; then the total amount of antibody administered is at a dose ranging from about 0.1 mg/kg to 18 mg/kg or from about 0.3 mg/kg to 18 mg/kg. Thus, in some embodiments, the dose administered is described as the combined amount of a first and second antibody administered to the subject. Thus, in some embodiments where e.g. 1 mg/kg of the first antibody is administered to the subject and 1 mg/kg of the second antibody is administered to the subject, the combined total amount of antibody administered to the subject is a dose of 2 mg/kg.

In one embodiment of the invention, the first and second antibody, or a pharmaceutically acceptable salt thereof, are administered at about a 49:1 to 1:49 molar ratio. In one embodiment of the invention, the first and second antibody, or a pharmaceutically acceptable salt thereof, are administered at about a 25:1 to 1:25 molar ratio. In one embodiment of the invention, the first and second antibody, or a pharmaceutically acceptable salt thereof, are administered at about a 15:1 to 1:15 molar ratio. In one embodiment of the invention, the first and second antibody, or a pharmaceutically acceptable salt thereof, are administered at about a 10:1 to 1:10 molar ratio. In one embodiment of the invention, the first and second antibody, or a pharmaceutically acceptable salt thereof, are administered at about a 5:1 to 1:5 molar ratio. In one embodiment of the invention, the first and second antibody, or a pharmaceutically acceptable salt thereof, are administered at about a 2:1 to 1:2 molar ratio. In one embodiment of the invention, the first and second antibody, or a pharmaceutically acceptable salt thereof, are administered at about a 1:1 molar ratio.

In one embodiment, the first and second antibody are combined with an immunomodulatory imide drug for an enhanced therapeutic effect.

In one embodiment of the invention, an immunomodulatory imide drug is administered to the subject prior to administration of the first and second antibody.

In one embodiment, the first and second antibody, and optionally the immunomodulatory imide drug are administered within the same treatment cycle as the first and second antibody.

In one embodiment, the immunomodulatory imide drug is administered based on a 28-days cycle. In one embodiment, the immunomodulatory imide drug is administered on each day from day 1 to 21 of a 28-days cycle.

In one embodiment, the immunomodulatory imide drug is administered by oral administration.

In one embodiment, the immunomodulatory imide drug is administered at a dose within the range of about 2.5 mg to 25 mg.

In one embodiment, the immunomodulatory imide drug is administered at a dose within the range of 2.5 mg to 25 mg. In one embodiment, the immunomodulatory imide drug is administered at a dose of about 2.5 mg. In one embodiment, the immunomodulatory imide drug is administered at a dose of about 5 mg. In one embodiment, the immunomodulatory imide drug is administered at a dose of about 10 mg. In one embodiment, the immunomodulatory imide drug is administered at a dose of about 15 mg. In one embodiment, the immunomodulatory imide drug is administered at a dose of about 25 mg. Hereby embodiments are described wherein the immunomodulatory imide drug is administered at a flat dose.

In one embodiment, the immunomodulatory imide drug is administered at a flat dose.

In the immunomodulatory imide drug is lenalidomide.

In one embodiment of the invention, lenalidomide is administered based on a 28-day cycle. In one embodiment, lenalidomide is administered on each day from day 1 to 21 of a 28-day cycle.

In one embodiment, lenalidomide is administered by oral administration.

In one embodiment, lenalidomide is administered on at a dose within the range of about 2.5 mg to 25 mg.

In one embodiment, lenalidomide is administered at a flat dose within the range of about 2.5 mg to 25 mg. In one embodiment, lenalidomide is administered at a flat dose of about 2.5 mg. In one embodiment, lenalidomide is administered at a flat dose of about 5 mg. In one embodiment, lenalidomide is administered at a flat dose of about 10 mg. In one embodiment, lenalidomide is administered at a flat dose of about 15 mg. In one embodiment, lenalidomide is administered at a flat dose of about 25mg.

In one embodiment of the invention, a steroid hormone is administered from three days prior to seven days after the administration of the first and second antibody. That is in one embodiment the steroid hormone is administered from day −3 to day 8, when the first and second antibody is administered on day 1 of a 14-day cycle. In one embodiment of the invention, a steroid hormone is administered one day to three days prior to the administration of the first and second antibody. In one embodiment of the invention, a steroid hormone is administered one day prior to the administration of the first and second antibody. In one embodiment of the invention, a steroid hormone is administered two days prior to the administration of the first and second antibody. In one embodiment of the invention, a steroid hormone is administered three days prior to the administration of the first and second antibody. In one embodiment of the invention, a steroid hormone is administered to the subject one the same day as the first and second antibody. In one embodiment of the invention, a steroid hormone is administered 1 day to 7 day following the administration of the first and second antibody. In one embodiment of the invention, a steroid hormone is administered 1 day to three days following the administration of the first and second antibody. The effect of administering the steroid hormone is to mitigate potential transaminase elevations caused by administration of the first and second antibody binding to DR5. Thus, administering a steroid may reduce, prevent or lessen the induction of transaminase levels by the first and second antibody, such as reduce, prevent or lessen the induction of alanine transaminase (ALT) or aspartate transaminase (AST).

In one embodiment of the invention, the steroid hormone is a corticosteroid. In one embodiment, the steroid hormone is dexamethasone.

In one embodiment of the invention, dexamethasone is administered to the subject from three days prior to 7 days after the administration of the first and second antibody. In one embodiment of the invention, dexamethasone is administered to the subject prior to administration of the first and second antibody. In one embodiment of the invention, dexamethasone is administered between one day to three days prior to the administration of the first and second antibody. In one embodiment of the invention, dexamethasone is administered one day prior to the administration of the first and second antibody. In one embodiment of the invention, dexamethasone is administered two days prior to the administration of the first and second antibody. In one embodiment of the invention, dexamethasone is administered three days prior to the administration of the first and second antibody. In one embodiment of the invention, dexamethasone is administered one the day of administration of the first and second antibody.

In one embodiment of the invention, dexamethasone is administered at a dose ranging from 1 to 100 mg. In one embodiment of the invention, dexamethasone is administered at a dose ranging from 5 to 20 mg. Thus, the dexamethasone is administered at a flat dose to the subject which does not depend on the weight of the subject. In one embodiment of the invention, dexamethasone is administered at a dose of 10 mg. Thus, in one embodiment of the invention, dexamethasone is administered at a dose of 10 mg per subject, where the dose administered does not depend on the weight of the subject. In one embodiment of the invention, dexamethasone is administered daily.

In one embodiment of the invention, dexamethasone is administered by intravenous infusion. In one embodiment of the invention, 10 mg dexamethasone is administered by intravenous infusion 1 day prior to the administration of the first and second antibody. Hereby embodiments are described wherein the dexamethasone is administered to mitigate transaminase elevations caused by administration of the first and second antibody binding to DR5. Thus, administering dexamethasone may reduce, prevent or lessen the induction of transaminase levels by the first and second antibody, such as reduce, prevent or lessen the induction of alanine transaminase (ALT) or aspartate transaminase (AST).

### Maintenance Therapy

A person of skill in the art, such as a physician, may determine that, after a suitable number of treatment cycles, the treatment cycles should be followed by maintenance therapy with a first and a second antibody binding to DR5, treatment with another therapeutic agent or combination of therapeutic agents, as appropriate.

In some embodiments, the subject will begin maintenance therapy following one or more, preferably two or more, such as following 3 or 4 or 5 or 6 or 7 or 8 or 9 or 10 or 11 or 12 or more cycles, such as 24 cycles or more, such as 36 cycles or more, of 7-days treatment cycles.

In some embodiments, the subject will begin maintenance therapy following one or more, preferably two or more, such as following 3 or 4 or 5 or 6 or 7 or 8 or 9 or 10 or 11 or 12 or more cycles, such as 24 cycles or more, such as 36 cycles or more, of 14-days treatment cycles.

In some embodiments, the subject will begin maintenance therapy following one or more, preferably two or more, such as following 3 or 4 or 5 or 6 or 7 or 8 or 9 or 10 or 11 or 12 or more cycles, such as 24 cycles or more, such as 36 cycles or more, of 28-days treatment cycles.

In some embodiments, the subject will start maintenance therapy following an evaluation indicating that the subject has reduced amount of cancer or no detectable cancer, e.g., following an evaluation indicating that the subject has had a complete response.

As used herein, “reduced administration frequency” refers to therapy with the first and second antibody binding DR5, but at a reduced administration schedule compared to an intensified dosing schedule where the antibody is dosed at e.g. once a week. During reduced administration frequency, the first and second antibody binding DR5 is preferably administered once every two weeks.

The first and second antibody binding to DR5 may alternatively be administered as a combination therapy. By the term “combination therapy” is meant that at least one other anti-cancer agent is administered to the subject during the treatment cycle with a first and second antibody binding to DR5. The first and second antibody binding to DR5 and the at least one other anti-cancer agent may be administered simultaneously and may optionally be provided in the same pharmaceutical composition. Typically, however, the first and second antibody binding to DR5 and the at least one other anti-cancer agent are separately administered and formulated as separate pharmaceutical compositions. For example, the at least one other anti-cancer agent may be administered according to the dosage regimen for which it has been approved by a medicines regulatory authority when administered as a monotherapy, or the at least one other anti-cancer agent may be administered according to a dosage regimen which is optimized for its combined use with the first and second antibody binding to DR5 as described herein.

The response to the anti-DR5 therapy may be evaluated by a person of skill in the art according to known methods, e.g., the guidelines of the NCCN or ESMO. In a specific embodiment, the evaluation can be based on the following criteria (IMWG criteria):

TABLE 1
Definition of Response (International Myeloma Working Group
(IMWG) Uniform Response Criteria for Multiple Myeloma)

| | |
|---|---|
| Response | |
| subcategory | Response criteria¹ |

IMWG criteria for response assessment including criteria for MRD
IMWG MRD criteria (requires a complete response as defined below)

| | |
|---|---|
| Sustained | MRD negativity in the marrow (NGF, NGS, or both) and by imaging as defined |
| MRD negative | below, confirmed minimum of 1 year apart. Subsequent evaluations can be |
| | used to further specify the duration of negativity (eg, MRD-negative at 5 |
| | years) |
| Flow MRD-negative | Absence of phenotypically aberrant clonal plasma cells by NGF³ on bone |
| | marrow aspirates using the EuroFlow standard operation procedure for MRD |
| | detection in multiple myeloma (or validated equivalent method) with a |
| | minimum sensitivity of 1 in 10⁵ nucleated cells or higher |
| Sequencing | Absence of clonal plasma cells by NGS or bone marrow aspirate in which |
| MRD-negative | presence of a clone is defined as less than two identical sequencing reads |
| | obtained after DNA sequencing of bone marrow aspirates using validated |
| | equivalent method with a minimum sensitivity of 1 in 10⁵ nucleated cells or |
| | higher |
| Imaging plus | MRD negativity as defined by NGF or NGS plus disappearance of every area of |
| MRD-negative | increased tracer uptake found at baseline or a preceding FDG PET/CT or |
| | decrease to less mediastinal blood pool standardized uptake value or decrease |
| | to less than that of surrounding normal tissue |

Standard IMWG response criteria

| | |
|---|---|
| sCR | CR as defined below plus |
| | Normal FLC ratio and |
| | Absence of clonal cells in bone marrow² by immunohistochemistry or |
| | immunofluorescence |
| CR | Negative immunofixation on the serum and urine and disappearance of any |
| | soft tissue plasmacytomas and ≤5% plasma cells in bone marrow² |
| VGPR | Serum and urine M-protein detectable by immunofixation but not on |
| | electrophoresis or 90% or greater reduction in serum M-protein plus urine M- |
| | protein level <100 mg per 24 h |
| PR | ≥50% reduction of serum M-protein and reduction in 24-h urinary M-protein |
| | by ≥90% or to <200 mg per 24 h |
| | If the serum and urine M-protein are unmeasurable, a ≥50% decrease in the |
| | difference between involved and uninvolved FLC levels is required in place of |
| | the M-protein criteria |
| | If serum and urine M-protein are unmeasurable, and serum free light assay is |
| | also unmeasurable, ≥50% reduction in plasma cells is required in place of M- |
| | protein, provided baseline bone marrow plasma cell percentage was ≥30% |
| | In addition to the above listed criteria, if present at baseline, a ≥50% |
| | reduction in the size of soft tissue plasmacytomas is also required |
| MR | ≥25% but ≤49% reduction of serum M-protein and reduction of 24-h urine by |
| | 50-89%. In addition to the above listed criteria, if present at baseline, a 24- |
| | 40% reduction in SPD of soft tissue plasmacytomas is also required. |
| SD | Not meeting criteria for CR, VGPR, PR, or PD |
| (not recommended | |
| for use as an | |
| indicator of response; | |
| stability of disease is | |
| best described by | |
| providing the time to | |
| progression estimates) | |
| PD⁴ | Increase of ≥25% from baseline in any one or more the following: |
| To be used for | Serum M-component and/or (the absolute increase must be ≥0.5 g/dL)² |
| calculation of time to | Urine M-component and/or (the absolute increase must be ≥200 mg/24 |
| progression and | hr. |
| progression-free | Only in subjects without measurable serum and urine M-protein levels: the |
| survival end points | difference between involved and uninvolved FLC levels. The absolute |
| for all subjects | increase must be >10 mg/dL. |
| including those in CR | Bone marrow plasma cell percentage: the absolute % must be >10%³ |
| (includes primary | Definite development of new bone lesions or soft tissue plasmacytomas or |
| progressive disease | definite |
| and disease | increase in the size of existing bone lesions or soft tissue plasmacytomas |
| progression on or off | Development of hypercalcemia (corrected serum calcium >11.5 mg/dL or |
| therapy) | 2.65 mmol/L) that can be attributed solely to the plasma cell proliferative |
| | disorder |
| Clinical Relapse⁴ | Clinical relapse requires one or more of the following: |
| | Direct indicators of increasing disease and/or end organ dysfunction (CRAB |
| | features).² It is not used in calculation of time to progression or progression- |
| | free survival but is listed here as something that can be reported optionally or |
| | for use in clinical practice: |
| | Development of new soft tissue plasmacytomas or bone lesions |
| | Definite increase in the size of existing plasmacytomas or bone lesions. A |
| | definite increase is defined as a 50% (and at least 1 cm) increase as |
| | measured serially by the sum of the products of the cross-diameters of the |
| | measurable lesion |
| | Hypercalcemia (>11.5 mg/dL) [2.65 mmol/L] |
| | Decrease in hemoglobin of ≥2 g/dL [1.25 mmol/L] |
| | Rise in serum creatinine by 2 mg/dL or more [177 μmol/L or more] |
| Relapse from CR⁴ | Any one or more of the following: |
| (To be used only if | Reappearance of serum or urine M-protein by immunofixation or |
| the end point studied | electrophoresis |
| is DFS)⁴ | Development of ≥5% plasma cells in the bone marrow³ |
| | Appearance of any other sign of progression (ie, new plasmacytoma, lytic |
| | bone lesion, or hypercalcemia) |
| Relapse from MRD | Any one or more of the following: |
| negative | Loss of MRD negative state (evidence of clonal plasma cells on NGF or |
| (To be used only if | NGS, or positive imaging study for recurrence of myeloma |
| the end point is DFS) | Reappearance of serum or urine M-protein by immunofixation or |
| | electrophoresis |
| | Development of ≥5% plasma cells in the bone marrow³ |
| | Appearance of any other sign of progression (ie, new plasmacytoma, lytic |
| | bone lesion, or hypercalcemia) |

CR = complete response; DFS = disease-free survival; CT = computed tomography; FDG = fluorodeoxyglucose; FLC = free light chain; MR = minimal response; MRD = minimal residual disease; NGF = next generation flow; NGS = next generation sequencing; PET = positron emission tomography; PR = partial response; sCR = stringent complete response; SD = stable disease; SPD = sum of the product of the diameters; VGPR = very good partial response.
¹All response categories require two consecutive assessments made at any time before the institution of any new therapy; all categories also require no known evidence of progressive or new bone lesions if radiographic studies were performed. Radiographic studies are not required to satisfy these response requirements.
²Confirmation with repeat bone marrow biopsy not needed.
³Presence/absence of clonal cells is based upon the k/λ ratio. An abnormal k/λ ratio by immunohistochemistry and/or immunofluorescence requires a minimum of 100 plasma cells for analysis. An abnormal ratio reflecting presence of an abnormal clone is k/λ of >44:1 or <1:2.
⁴All relapse categories require two consecutive assessments made at any time before classification as relapse or disease progression and/or the institution of any new therapy.
⁵For progressive disease, serum M-component increases of ≥1 gm/dl are sufficient to define relapse if starting M-component is ≥5 g/dl.
⁶Relapse from CR has the 5% cutoff versus 10% for other categories of relapse.
⁷For purposes of calculating time to progression and progression-free survival, subjects with CR should also be evaluated using criteria listed above for progressive disease.
Source: NCCN (2019). NCCN Clinical Practice Guidelines in Oncology. Multiple Myeloma. Version 2.2020.

### Pharmaceutical Compositions

In another aspect of the invention, the first and/or second antibody binding DR5 for use according to any aspect or embodiment of the invention as described herein is/are comprised in a pharmaceutical composition. In one embodiment, the pharmaceutical composition further comprises a pharmaceutically acceptable carrier. In particular, upon purifying the first and/or second antibody binding DR5, they may be formulated into pharmaceutical compositions using well known pharmaceutical carriers or excipients.

The pharmaceutical compositions may be formulated with pharmaceutically acceptable carriers or diluents as well as any known adjuvants and excipients in accordance with conventional techniques such as those disclosed in Remington: The Science and Practice of Pharmacy, 19th Edition, Gennaro, Ed., Mack Publishing Co., Easton, PA, 1995.

The pharmaceutically acceptable carriers or diluents as well as any known adjuvants and excipients should be suitable for the antibodies of the present invention and the chosen mode of administration. Suitability for carriers and other components of pharmaceutical compositions is determined based on the lack of significant negative effect on the desired biological properties of the compound or pharmaceutical composition of the present invention (e.g., less than a substantial effect (10% or less relative inhibition, 5% or less relative inhibition, etc.)) on antigen binding.

A pharmaceutical composition of the present invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The pharmaceutical composition may be administered by any suitable route and mode. Suitable routes of administering an antibody of the present invention are well-known in the art and may be selected by those of ordinary skill in the art.

In one embodiment, the pharmaceutical composition of the present invention is administered by intravenous administration.

In one embodiment, the pharmaceutical composition of the present invention is administered by intravenous infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with the antibodies of the present invention.

Examples of suitable aqueous-and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the first and/or second antibody of the present invention, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

The pharmaceutical compositions of the present invention may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palm itate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride.

The pharmaceutical compositions of the present invention may also contain one or more adjuvants appropriate for the chosen route of administration such as preservatives, wetting agents, emulsifying agents, dispersing agents or buffers, which may prolong the shelf life or effectiveness of the pharmaceutical composition. The first and/or second antibody binding DR5 of the present invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J.R. Robinson, ed., Marcel Dekker, Inc., New York, 1978.

In one embodiment, the first and/or second antibody binding DR5 of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the first and/or second antibody binding DR5 in the required amount in an appropriate solvent with one or a combination of ingredients e.g. as enumerated above, as required, followed by sterilization microfiltration. Generally, dispersions are prepared by incorporating the first and/or second antibody binding DR5 into a sterile vehicle that contains a basic dispersion medium and the required other ingredients e.g. from those enumerated above.

Sterile injectable solutions may be prepared by incorporating the first and/or second antibody binding DR5 in the required amount in an appropriate solvent with one or a combination of ingredients enumerated above, as required, followed by sterilization microfiltration. Generally, dispersions are prepared by incorporating the first and/or second antibody binding DR5 into a sterile vehicle that contains a basic dispersion medium and the required other ingredients from those enumerated above.

In one particular embodiment, the first and/or second antibody binding DR5 is comprised in a pharmaceutical composition which comprises one or more excipients but is free of surfactant. In one embodiment, the pharmaceutical composition has a pH of about 5.5 to about 7 and comprises, in aqueous solution:
(a) from about 5 mg/mL to about 30 mg/mL of a first and second antibody binding to DR5;
(b) histidine; and
(c) sodium chloride.

In one embodiment of the invention, the pharmaceutical composition has a pH of about 6.

In a specific embodiment, the pharmaceutical composition has a pH in the range of about 5.5 to about 6.5 and comprises:
(a) from about 2 mg/mL to about 20 mg/mL of a first and second antibody binding DR5, such as about 10 mg/mL of the first antibody binding DR5 and 10 mg/mL of the second antibody binding DR5;
(b) from about 10 mM to about 50 mM histidine, such as about 30 mM histidine;
(c) from about 50 mM to 250 mM sodium chloride , such as about 150 mM sodium chloride.

In one embodiment of the invention, the pharmaceutical composition has a pH of about 6 and comprises:
(a) 10 mg/mL of the first antibody binding DR5 and 10 mg/mL of the second antibody binding DR5;
(b) from about 30 mM histidine; and
(c) 150 mM sodium chloride.

In one embodiment of the invention, the pharmaceutical composition has a pH of about 6 and comprises:
(a) 20 mg/mL of the first antibody binding DR5 and 20 mg/mL of the second antibody binding DR5;
(b) from about 30 mM histidine; and
(c) 150 mM sodium chloride.

A further aspect of the invention provides a kit of parts comprising a first antibody capable of binding DR5 and a second antibody capable of binding DR5, or a pharmaceutically acceptable salt thereof, and an immunomodulatory imide drug. Further instruction for use may be included.

The first and second antibody capable of binding DR5 to be included in such a kit may characterized as any first and second antibody described herein. That is the first and second antibody may be as defined above.

Further, in the kit according to the invention the immunomodulatory imide drug is as defined above.

TABLE 2

| SEQ ID NO | Name | Sequence | Remarks |
|---|---|---|---|
| SEQ ID No: 1 | VH hDR5-01-G56T | GFNIKDTF | IgG1-hDR5-01- |
| | CDR1 | | G56T-E430G |
| SEQ ID No: 2 | VH hDR5-01-G56T | IDPANTNT | |
| | CDR2 | | |
| SEQ ID No: 3 | VH hDR5-01-G56T | VRGLYTYYFDY | |
| | CDR3 | | |
| SEQ ID No: 4 | VH hDR5-01-G56T | EVQLQQSGAEVVKPGASVKLSCKASGFNIKDTFIHWVK | |
| | | QAPGQGLEWIGRIDPANTNTKYDPKFQGKATITTDTSS | |
| | | NTAYMELSSLRSEDTAVYYCVRGLYTYYFDYWGQGTL | |
| | | VTVSS | |
| SEQ ID No: 5 | VL hDR5-01-G56T CDR1 | QSISNN | |
| | VL hDR5-01-G56T CDR2 | FAS | |
| SEQ ID No: 6 | VL hDR5-01-G56T CDR3 | QQGNSWPYT | |
| SEQ ID No: 7 | VL hDR5-01-G56T | EIVMTQSPATLSVSPGERATLSCRASQSISNNLHWYQQ | |
| | | KPGQAPRLLIKFASQSITGIPARFSGSGSGTEFTLTIS | |
| | | SLQSEDFAVYYCQQGNSWPYTFGQGTKLEIK | |
| SEQ ID No: 8 | VH hDR5-05 CDR1 | GFNIKDTH | IgG1-hDR5-05- |
| | | | E430G |
| SEQ ID No: 9 | VH hDR5-05 CDR2 | IDPANGNT | |
| SEQ ID No: 10 | VH hDR5-05 CDR3 | ARWGTNVYFAY | |
| SEQ ID No: 11 | VH hDR5-05 | QVQLVQSGAEVKKPGASVKVSCKASGFNIKDTHMHW | |
| | | VRQAPGQRLEWIGRIDPANGNTEYDQKFQGRVTITVD | |
| | | TSASTAYMELSSLRSEDTAVYYCARWGTNVYFAYWGQ | |
| | | GTLVTVSS | |
| SEQ ID No: 12 | VL hDR5-05 CDR1 | SSVSY | |
| | VL hDR5-05 CDR2 | RTS | |
| SEQ ID No: 13 | VL hDR5-05 CDR3 | QQYHSYPPT | |
| SEQ ID No: 14 | VL hDR5-05 | DIQLTQSPSSLSASVGDRVTITCSASSSVSYMYWYQQK | |
| | | PGKAPKPWIYRTSNLASGVPSRFSGSGSGTDFTLTISS | |
| | | LQPEDFATYYCQQYHSYPPTFGGGTKVEIK | |
| SEQ ID No: 15 | Fc IgG1m(f) | ASTKGPSVFPLAPSSKSTSGGTAALGCLVKDYFPEPVTV | |
| | | SWNSGALTSGVHTFPAVLQSSGLYSLSSVVTVPSSSLGT | |
| | | QTYICNVNHKPSNTKVDKRVEPKSCDKTHTCPPCPAPE | |
| | | LLGGPSVFLFPPKPKDTLMISRTPEVTCVVVDVSHEDPE | |
| | | VKFNWYVDGVEVHNAKTKPREEQYNSTYRVVSVLTVL | |
| | | HQDWLNGKEYKCKVSNKALPAPIEKTISKAKGQPREP | |
| | | QVYTLPPSREEMTKNQVSLTCLVKGFYPSDIAVEWESN | |
| | | GQPENNYKTTPPVLDSDGSFFLYSKLTVDKSRWQQGN | |
| | | VFSCSVMHEALHNHYTQKSLSLSPGK | |
| SEQ ID No:16 | Fc IgG1m(f)-E430G | ASTKGPSVFPLAPSSKSTSGGTAALGCLVKDYFPEPVTV | |
| | | SWNSGALTSGVHTFPAVLQSSGLYSLSSVVTVPSSSLGT | |
| | | QTYICNVNHKPSNTKVDKRVEPKSCDKTHTCPPCPAPE | |
| | | LLGGPSVFLFPPKPKDTLMISRTPEVTCVVVDVSHEDPE | |
| | | VKFNWYVDGVEVHNAKTKPREEQYNSTYRVVSVLTVL | |
| | | HQDWLNGKEYKCKVSNKALPAPIEKTISKAKGQPREP | |
| | | QVYTLPPSREEMTKNQVSLTCLVKGFYPSDIAVEWESN | |
| | | GQPENNYKTTPPVLDSDGSFFLYSKLTVDKSRWQQGN | |
| | | VFSCSVMHGALHNHYTQKSLSLSPGK | |
| SEQ ID No: 17 | HC hDR5-01-G56T- | EVQLQQSGAEVVKPGASVKLSCKASGFNIKDTFIHWVK | IgG1-hDR5-01- |
| | E430G | QAPGQGLEWIGRIDPANTNTKYDPKFQGKATITTDTSS | G56T-E430G |
| | | NTAYMELSSLRSEDTAVYYCVRGLYTYYFDYWGQGTL | |
| | | VTVSSASTKGPSVFPLAPSSKSTSGGTAALGCLVKDYFP | |
| | | EPVTVSWNSGALTSGVHTFPAVLQSSGLYSLSSVVTVP | |
| | | SSSLGTQTYICNVNHKPSNTKVDKRVEPKSCDKTHTCP | |
| | | PCPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDIA | |
| | | VEWESNGQPENNYKTTPPVLDSDGSFFLYSKLTVDKSR | |
| | | WQQGNVFSCSVMHGALHNHYTQKSLSLSPGK | |
| SEQ ID No: 18 | HC hDR5-01-G56T- | EVQLQQSGAEVVKPGASVKLSCKASGFNIKDTFIHWVK | IgG1-hDR5-01- |
| | E430G | QAPGQGLEWIGRIDPANTNTKYDPKFQGKATITTDTSS | G56T-E430G |
| | | NTAYMELSSLRSEDTAVYYCVRGLYTYYFDYWGQGTL | Without C- |
| | | VTVSSASTKGPSVFPLAPSSKSTSGGTAALGCLVKDYFP | terminal lysine |
| | | EPVTVSWNSGALTSGVHTFPAVLQSSGLYSLSSVVTVP | (K) |
| | | SSSLGTQTYICNVNHKPSNTKVDKRVEPKSCDKTHTCP | |
| | | PCPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDIA | |
| | | VEWESNGQPENNYKTTPPVLDSDGSFFLYSKLTVDKSR | |
| | | WQQGNVFSCSVMHGALHNHYTQKSLSLSPG | |
| SEQ ID No: 19 | LC hDR5-01-G56T- | EIVMTQSPATLSVSPGERATLSCRASQSISNNLHWYQQ | IgG1-hDR5-01- |
| | E430G | KPGQAPRLLIKFASQSITGIPARFSGSGSGTEFTLTISSLQ | G56T-E430G |
| | | SEDFAVYYCQQGNSWPYTFGQGTKLEIKRTVAAPSVFI | |
| | | FPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| SEQ ID No: 20 | HC hDR5-05-E430G | QVQLVQSGAEVKKPGASVKVSCKASGFNIKDTHMHW | IgG1-hDR5-05- |
| | | VRQAPGQRLEWIGRIDPANGNTEYDQKFQGRVTITVD | E430G |
| | | TSASTAYMELSSLRSEDTAVYYCARWGTNVYFAYWGQG | |
| | | TLVTVSSASTKGPSVFPLAPSSKSTSGGTAALGCLVKD | |
| | | YFPEPVTVSWNSGALTSGVHTFPAVLQSSGLYSLSSVV | |
| | | TVPSSSLGTQTYICNVNHKPSNTKVDKRVEPKSCDKTH | |
| | | TCPPCPAPELLGGPSVFLFPPKPKDTLMISRTPEVTCV | |
| | | VVDVSHEDPEVKFNWYVDGVEVHNAKTKPREEQYNSTY | |
| | | RVVSVLTVLHQDWLNGKEYKCKVSNKALPAPIEKTISK | |
| | | AKGQPREPQVYTLPPSREEMTKNQVSLTCLVKGFYPSD | |
| | | IAVEWESNGQPENNYKTTPPVLDSDGSFFLYSKLTVDK | |
| | | SRWQQGNVFSCSVMHGALHNHYTQKSLSLSPGK | |
| SEQ ID No: 21 | HC hDR5-05-E430G | QVQLVQSGAEVKKPGASVKVSCKASGFNIKDTHMHW | IgG1-hDR5-05- |
| | | VRQAPGQRLEWIGRIDPANGNTEYDQKFQGRVTITVD | E430G |
| | | TSASTAYMELSSLRSEDTAVYYCARWGTNVYFAYWGQ | Without C- |
| | | GTLVTVSSASTKGPSVFPLAPSSKSTSGGTAALGCLVKD | terminal lysine |
| | | YFPEPVTVSWNSGALTSGVHTFPAVLQSSGLYSLSSVV | (K) |
| | | TVPSSSLGTQTYICNVNHKPSNTKVDKRVEPKSCDKTH | |
| | | TCPPCPAPELLGGPSVFLFPPKPKDTLMISRTPEVTCVV | |
| | | VDVSHEDPEVKFNWYVDGVEVHNAKTKPREEQYNST | |
| | | YRVVSVLTVLHQDWLNGKEYKCKVSNKALPAPIEKTISK | |
| | | AKGQPREPQVYTLPPSREEMTKNQVSLTCLVKGFYPSD | |
| | | IAVEWESNGQPENNYKTTPPVLDSDGSFFLYSKLTVDK | |
| | | SRWQQGNVFSCSVMHGALHNHYTQKSLSLSPG | |
| SEQ ID No: 22 | LC hDR5-05-E430G | DIQLTQSPSSLSASVGDRVTITCSASSSVSYMYWYQQK | IgG1-hDR5-05- |
| | | PGKAPKPWIYRTSNLASGVPSRFSGSGSGTDFTLTISSL | E430G |
| | | QPEDFATYYCQQYHSYPPTFGGGTKVEIKRTVAAPSVFI | |
| | | FPPSDEQLKSGTASVVCLLNNFYPREAKVQWKVDNALQ | |
| | | SGNSQESVTEQDSKDSTYSLSSTLTLSKADYEKHKVYA | |
| | | CEVTHQGLSSPVTKSFNRGEC | |
| SEQ ID No: 1 | VH DR5-01 CDR1 | GFNIKDTF | mouse IgG1- |
| | | | DR5-01 |
| SEQ ID No: 9 | VH DR5-01 CDR2 | IDPANGNT | |
| SEQ ID No: 3 | VH DR5-01 CDR3 | VRGLYTYYFDY | |
| SEQ ID No: 23 | VH DR5-01 | EVQLQQSGAEFVKPGASVKLSCTASGFNIKDTFIHWVK | |
| | | QRPEQGLDWIGRIDPANGNTKYDPKFQGKATETTDTS | |
| | | SNTAYLQLSSLTSEDTAVYYCVRGLYTYYFDYWGQGTS | |
| | | VTVSS | |
| SEQ ID No: 5 | VL DR5-01 CDR1 | QSISNN | |
| | VL DR5-01 CDR2 | FAS | |
| SEQ ID No: 6 | VL DR5-01 CDR3 | QQGNSWPYT | |
| SEQ ID No: 24 | VL DR5-01 | DIVLTQSPATLSVTPGDSVSLSCRASQSISNNLHWYQQ | |
| | | KSHESPRLLIKFASQSISGIPSRFSGSGSGADFTLIIN | |
| | | SVETEDFGMYFCQQGNSWPYTFGGGTKLEIK | |
| SEQ ID No: 25 | HC DR5-01-K409R- | EVQLQQSGAEFVKPGASVKLSCTASGFNIKDTFIHWVK | Chimeric IgG1- |
| | E430G | QRPEQGLDWIGRIDPANGNTKYDPKFQGKATETTDTS | DR5-01-K409R- |
| | | SNTAYLQLSSLTSEDTAVYYCVRGLYTYYFDYWGQGTS | E430G |
| | | VTVSSASTKGPSVFPLAPSSKSTSGGTAALGCLVKDYFP | |
| | | EPVTVSWNSGALTSGVHTFPAVLQSSGLYSLSSVVTVP | |
| | | SSSLGTQTYICNVNHKPSNTKVDKRVEPKSCDKTHTCP | |
| | | PCPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDIA | |
| | | VEWESNGQPENNYKTTPPVLDSDGSFFLYSRLTVDKSR | |
| | | WQQGNVFSCSVMHGALHNHYTQKSLSLSPGK | |
| SEQ ID No: 26 | LC DR5-01-K409R- | DIVLTQSPATLSVTPGDSVSLSCRASQSISNNLHWYQQ | |
| | E430G | KSHESPRLLIKFASQSISGIPSRFSGSGSGADFTLIINSVET | |
| | | EDFGMYFCQQGNSWPYTFGGGTKLEIKRTVAAPSVFI | |
| | | FPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| SEQ ID No: 8 | VH DR5-05 CDR1 | GFNIKDTH | Mouse IgG1- |
| | | | DR5-05 |
| SEQ ID No: 9 | VH DR5-05 CDR2 | IDPANGNT | |
| SEQ ID No: 10 | VH DR5-05 CDR3 | ARWGTNVYFAY | |
| SEQ ID No: 27 | VH DR5-05 | EVQLQQSGAELVKPGASVKLFCTASGFNIKDTHIHWV | |
| | | KQRPEQGLEWIGRIDPANGNTEYDPKFQGKATIRVDT | |
| | | SSDTAYLQLSSLTSEDTAVHYCARWGTNVYFAYWGQ | |
| | | GTSVTVSS | |
| SEQ ID No: 12 | VL DR5-05 CDR1 | SSVSY | |
| | VL DR5-05 CDR2 | RTS | |
| SEQ ID No: 13 | VL DR5-05 CDR3 | QQYHSYPPT | |
| SEQ ID No: 28 | VL DR5-05 | DIQLTQSPAIMSASPGEKVTISCSASSSVSYMYWYQQK | |
| | | PGSSPKPWIYRTSNLASGVPGRFSGSGSGTSYSLTISSM | |
| | | EAEDAATYYCQQYHSYPPTFGGGTKLEIK | |
| SEQ ID No: 29 | HC DR5-05-F405L- | EVQLQQSGAELVKPGASVKLFCTASGFNIKDTHIHWVK | chimeric IgG1- |
| | E430G | QRPEQGLEWIGRIDPANGNTEYDPKFQGKATIRVDTS | DR5-05-F405L- |
| | | SDTAYLQLSSLTSEDTAVHYCARWGTNVYFAYWGQGT | E430G |
| | | SVTVSSASTKGPSVFPLAPSSKSTSGGTAALGCLVKDYF | |
| | | PEPVTVSWNSGALTSGVHTFPAVLQSSGLYSLSSVVTV | |
| | | PSSSLGTQTYICNVNHKPSNTKVDKRVEPKSCDKTHTC | |
| | | PPCPAPELLGGPSVFLFPPKPKDTLMISRTPEVTCVVVD | |
| | | VSHEDPEVKFNWYVDGVEVHNAKTKPREEQYNSTYR | |
| | | VVSVLTVLHQDWLNGKEYKCKVSNKALPAPIEKTISKA | |
| | | KGQPREPQVYTLPPSREEMTKNQVSLTCLVKGFYPSDI | |
| | | AVEWESNGQPENNYKTTPPVLDSDGSFLLYSKLTVDKS | |
| | | RWQQGNVFSCSVMHGALHNHYTQKSLSLSPGK | |
| SEQ ID No: 30 | LC DR5-05-F405L- | DIQLTQSPAIMSASPGEKVTISCSASSSVSYMYWYQQK | |
| | E430G | PGSSPKPWIYRTSNLASGVPGRFSGSGSGTSYSLTISSM | |
| | | EAEDAATYYCQQYHSYPPTFGGGTKLEIKRTVAAPSVFI | |
| | | FPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| SEQ ID No: 31 | VH b12 CDR1 | GYRFSNFV | IgG1-b12 |
| SEQ ID No: 32 | VH b12 CDR2 | INPYNGNK | |
| SEQ ID No: 33 | VH b12 CDR3 | ARVGPYSWDDSPQDNYYMDV | |
| SEQ ID No: 34 | VH b12 | QVQLVQSGAEVKKPGASVKVSCQASGYRFSNFVIHWV | |
| | | RQAPGQRFEWMGWINPYNGNKEFSAKFQDRVTFTA | |
| | | DTSANTAYMELRSLRSADTAVYYCARVGPYSWDDSPQ | |
| | | DNYYMDVWGKGTTVIVSS | |
| SEQ ID No: 35 | VL b12 CDR1 | HSIRSRR | |
| | VL b12 CDR2 | GVS | |
| SEQ ID No: 36 | VL b12 CDR3 | QVYGASSYT | |
| SEQ ID No: 37 | VL b12 | EIVLTQSPGTLSLSPGERATFSCRSSHSIRSRRVAWYQH | |
| | | KPGQAPRLVIHGVSNRASGISDRFSGSGSGTDFTLTITR | |
| | | VEPEDFALYYCQVYGASSYTFGQGTKLERK | |
| SEQ ID No: 38 | HC b12 | QVQLVQSGAEVKKPGASVKVSCQASGYRFSNFVIHWV | |
| | | RQAPGQRFEWMGWINPYNGNKEFSAKFQDRVTFTA | |
| | | DTSANTAYMELRSLRSADTAVYYCARVGPYSWDDSPQ | |
| | | DNYYMDVWGKGTTVIVSSASTKGPSVFPLAPSSKSTSG | |
| | | GTAALGCLVKDYFPEPVTVSWNSGALTSGVHTFPAVL | |
| | | QSSGLYSLSSVVTVPSSSLGTQTYICNVNHKPSNTKVDK | |
| | | RVEPKSCDKTHTCPPCPAPELLGGPSVFLFPPKPKDTL | |
| | | MISRTPEVTCVVVDVSHEDPEVKFNWYVDGVEVHNA | |
| | | KTKPREEQYNSTYRVVSVLTVLHQDWLNGKEYKCKVS | |
| | | NKALPAPIEKTISKAKGQPREPQVYTLPPSREEMTKNQ | |
| | | VSLTCLVKGFYPSDIAVEWESNGQPENNYKTTPPVLDS | |
| | | DGSFFLYSKLTVDKSRWQQGNVFSCSVMHEALHNHY | |
| | | TQKSLSLSPGK | |
| SEQ ID No: 39 | LC b12 | EIVLTQSPGTLSLSPGERATFSCRSSHSIRSRRVAWYQH | |
| | | KPGQAPRLVIHGVSNRASGISDRFSGSGSGTDFTLTITR | |
| | | VEPEDFALYYCQVYGASSYTFGQGTKLERKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC | |
| SEQ ID No: 40 | HC b12-E430G | QVQLVQSGAEVKKPGASVKVSCQASGYRFSNFVIHWV | IgG1-b12- |
| | | RQAPGQRFEWMGWINPYNGNKEFSAKFQDRVTFTA | E430G |
| | | DTSANTAYMELRSLRSADTAVYYCARVGPYSWDDSPQ | |
| | | DNYYMDVWGKGTTVIVSSASTKGPSVFPLAPSSKSTSG | |
| | | GTAALGCLVKDYFPEPVTVSWNSGALTSGVHTFPAVL | |
| | | QSSGLYSLSSVVTVPSSSLGTQTYICNVNHKPSNTKVDK | |
| | | RVEPKSCDKTHTCPPCPAPELLGGPSVFLFPPKPKDTL | |
| | | MISRTPEVTCVVVDVSHEDPEVKFNWYVDGVEVHNA | |
| | | KTKPREEQYNSTYRVVSVLTVLHQDWLNGKEYKCKVS | |
| | | NKALPAPIEKTISKAKGQPREPQVYTLPPSREEMTKNQ | |
| | | VSLTCLVKGFYPSDIAVEWESNGQPENNYKTTPPVLDS | |
| | | DGSFFLYSKLTVDKSRWQQGNVFSCSVMHGALHNHY | |
| | | TQKSLSLSPGK | |
| SEQ ID No: 39 | LC b12-E430G | EIVLTQSPGTLSLSPGERATFSCRSSHSIRSRRVAWYQH | |
| | | KPGQAPRLVIHGVSNRASGISDRFSGSGSGTDFTLTITR | |
| | | VEPEDFALYYCQVYGASSYTFGQGTKLERKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC | |
| SEQ ID No: 41 | Human DR5 | MEQRGQNAPAASGARKRHGPGPREARGARPGPRVP | Human DR5 |
| | (UniProt 014763-1) | KTLVLVVAAVLLLVSAESALITQQDLAPQQRAAPQQKR | isoform long |
| | | SSPSEGLCPPGHHISEDGRDCISCKYGQDYSTHWNDLL | |
| | | FCLRCTRCDSGEVELSPCTTTRNTVCQCEEGTFREEDSP | |
| | | EMCRKCRTGCPRGMVKVGDCTPWSDIECVHKESGTK | |
| | | HSGEVPAVEETVTSSPGTPASPCSLSGIIIGVTVAAVVLI | |
| | | VAVFVCKSLLWKKVLPYLKGICSGGGGDPERVDRSSQR | |
| | | PGAEDNVLNEIVSILQPTQVPEQEMEVQEPAEPTGVN | |
| | | MLSPGESEHLLEPAEAERSQRRRLLVPANEGDPTETLR | |
| | | QCFDDFADLVPFDSWEPLMRKLGLMDNEIKVAKAEA | |
| | | AGHRDTLYTMLIKWVNKTGRDASVHTLLDALETLGERL | |
| | | AKQKIEDHLLSSGKFMYLEGNADSAMS | |
| SEQ ID No: 42 | Human DR5 natural | MEQRGQNAPAASGARKRHGPGPREARGARPGLRVP | SEQ ID No: 41 |
| | variant (Accession: | KTLVLVVAAVLLLVSAESALITQQDLAPQQRVAPQQKR | with naturally |
| | AAB70578) | SSPSEGLCPPGHHISEDGRDCISCKYGQDYSTHWNDLL | occurring P32L; |
| | | FCLRCTRCDSGEVELSPCTTTRNTVCQCEEGTFREEDSP | A67V; V191A |
| | | EMCRKCRTGCPRGMVKVGDCTPWSDIECVHKESGTK | substitutions |
| | | HSGEAPAVEETVTSSPGTPASPCSLSGIIIGVTVAAVVLI | |
| | | VAVFVCKSLLWKKVLPYLKGICSGGGGDPERVDRSSQR | |
| | | PGAEDNVLNEIVSILQPTQVPEQEMEVQEPAEPTGVN | |
| | | MLSPGESEHLLEPAEAERSQRRRLLVPANEGDPTETLR | |
| | | QCFDDFADLVPFDSWEPLMRKLGLMDNEIKVAKAEA | |
| | | AGHRDTLYTMLIKWVNKTGRDASVHTLLDALETLGERL | |
| | | AKQKIEDHLLSSGKFMYLEGNADSAMS | |
| SEQ ID No: 43 | Human DR5 | MEQRGQNAPAASGARKRHGPGPREARGARPGPRVP | Human DR5 |
| | (UniProt 014763-2) | KTLVLVVAAVLLLVSAESALITQQDLAPQQRAAPQQKR | isoform short: |
| | | SSPSEGLCPPGHHISEDGRDCISCKYGQDYSTHWNDLL | missing aa 185- |
| | | FCLRCTRCDSGEVELSPCTTTRNTVCQCEEGTFREEDSP | 213 compared |
| | | EMCRKCRTGCPRGMVKVGDCTPWSDIECVHKESGIII | to UniProt |
| | | GVTVAAVVLIVAVFVCKSLLWKKVLPYLKGICSGGGGD | 014763-1 in |
| | | PERVDRSSQRPGAEDNVLNEIVSILQPTQVPEQEMEV | SEQ ID No: 41 |
| | | QEPAEPTGVNMLSPGESEHLLEPAEAERSQRRRLLVPA | |
| | | NEGDPTETLRQCFDDFADLVPFDSWEPLMRKLGLMD | |
| | | NEIKVAKAEAAGHRDTLYTMLIKWVNKTGRDASVHTL | |
| | | LDALETLGERLAKQKIEDHLLSSGKFMYLEGNADSAMS | |
| SEQ ID No: 44 | Human DR5del-K386N | MEQRGQNAPAASGARKRHGPGPREARGARPGPRVP | Human DR5 |
| | | KTLVLVVAAVLLLVSAESALITQQDLAPQQRAAPQQKR | (based on |
| | | SSPSEGLCPPGHHISEDGRDCISCKYGQDYSTHWNDLL | Uniprot |
| | | FCLRCTRCDSGEVELSPCTTTRNTVCQCEEGTFREEDSP | 014763-2 in |
| | | EMCRKCRTGCPRGMVKVGDCTPWSDIECVHKESGIII | SEQ ID No: 43) |
| | | GVTVAAVVLIVAVFVCKSLLWKKVLPYLKGICSGGGGD | with K386N |
| | | PERVDRSSQRPGAEDNVLNEIVSILQPTQVPEQEMEV | mutation as |
| | | QEPAEPTGVNMLSPGESEHLLEPAEAERSQRRRLLVPA | used in the |
| | | NEGDPTETLRQCFDDFADLVPFDSWEPLMRKLGLMD | examples |
| | | NEIKVAKAEAAGHRDTLYTMLIKWVNKTGRDASVHTL | |
| | | LDALETLGERLANQKIEDHLLSSGKFMYLEGNADSAMS | |
| SEQ ID No: 45 | Cynomolgus monkey | MGQLRQSAPAASGARKGRGPGPREARGARPGLRVLK | |
| | DR5 | TLVLVVAAARVLLSVSADCAPITRQSLDPQRRAAPQQK | |
| | (NCBI | RSSPTEGLCPPGHHISEDSRECISCKYGQDYSTHWNDF | |
| | XP_005562887.1) | LFCLRCTKCDSGEVEVNSCTTTRNTVCQCEEGTFREED | |
| | | SPEICRKCRTGCPRGMVKVKDCTPWSDIECVHKESGTK | |
| | | HTGEVPAVEKTVTTSPGTPASPCSLSGIIIGVIVLVVIVVV | |
| | | AVIVWKTSLWKKVLPYLKGVCSGGGGDPERVDSSSHS | |
| | | PQRPGAEDNALNEIVSIVQPSQVPEQEMEVQEPAEQT | |
| | | DVNTLSPGESEHLLEPAKAEGPQRRGQLVPVNENDPT | |
| | | ETLRQCFDDFAAIVPFDAWEPLVRQLGLTNNEIKVAKA | |
| | | EAASSRDTLYVMLIKWVNKTGRAASVNTLLDALETLEE | |
| | | RLAKQKIQDRLLSSGKFMYLEDNADSATS | |
| SEQ ID No: 46 | Cyno DR5Mfdel-K420N | MGQLRQSAPAASGARKGRGPGPREARGARPGLRVLK | Cynomolgus |
| | | TLVLVVAAARVLLSVSADCAPITRQSLDPQRRAAPQQK | monkey DR5 as |
| | | RSSPTEGLCPPGHHISEDSRECISCKYGQDYSTHWNDF | in SEQ ID No: |
| | | LFCLRCTKCDSGEVEVNSCTTTRNTVCQCEEGTFREED | 45 with |
| | | SPEICRKCRTGCPRGMVKVKDCTPWSDIECVHKESGIII | deletion of aa |
| | | GVIVLVVIVVVAVIVWKTSLWKKVLPYLKGVCSGGGGD | 185-213 and |
| | | PERVDSSSHSPQRPGAEDNALNEIVSIVQPSQVPEQE | K420N |
| | | MEVQEPAEQTDVNTLSPGESEHLLEPAKAEGPQRRGQ | mutation as |
| | | LVPVNENDPTETLRQCFDDFAAIVPFDAWEPLVRQLG | used in the |
| | | LTNNEIKVAKAEAASSRDTLYVMLIKWVNKTGRAASV | examples |
| | | NTLLDALETLEERLANQKIQDRLLSSGKFMYLEDNADS | |
| | | ATS | |
| SEQ ID No: 47 | Murine DR5 | MEPPGPSTPTASAAARADHYTPGLRPLPKRRLLYSFALL | |
| | (Uniprot Q9QZM4) | LAVLQAVFVPVTANPAHNRPAGLQRPEESPSRGPCLA | |
| | | GQYLSEGNCKPCREGIDYTSHSNHSLDSCILCTVCKEDK | |
| | | VVETRCNITTNTVCRCKPGTFEDKDSPEICQSCSNCTD | |
| | | GEEELTSCTPRENRKCVSKTAWASWHKLGLWIGLLVP | |
| | | VVLLIGALLVWKTGAWRQWLLCIKRGCERDPESANSV | |
| | | HSSLLDRQTSSTTNDSNHNTEPGKTQKTGKKLLVPVNG | |
| | | NDSADDLKFIFEYCSDIVPFDSWNRLMRQLGLTDNQI | |
| | | QMVKAETLVTREALYQMLLKWRHQTGRSASINHLLD | |
| | | ALEAVEERDAMEKIEDYAVKSGRFTYQNAAAQPETGP | |
| | | GGSQCV | |
| SEQ ID No: 48 | DR5ECD-FcRbHisCtag | MEQRGQNAPAASGARKRHGPGPREARGARPGLRVP | Extracellular |
| | | KTLVLVVAAVLLLVSAESALITQQDLAPQQRVAPQQKR | domain of |
| | | SSPSEGLCPPGHHISEDGRDCISCKYGQDYSTHWNDLL | human DR5 |
| | | FCLRCTRCDSGEVELSPCTTTRNTVCQCEEGTFREEDSP | natural variant |
| | | EMCRKCRTGCPRGMVKVGDCTPWSDIECVHKESGTK | P32L; A67V; |
| | | HSGEAPAVEETVTSSPGTPASPCSAPSTCSKPTCPPPEL | V191A fused to |
| | | LGGPSVFIFPPKPKDTLMISRTPEVTCVVVDVSQDDPE | rabbit Fc |
| | | VQFTWYINNEQVRTARPPLREQQFNSTIRVVSTLPIAH | domain, His tag |
| | | QDWLRGKEFKCKVHNKALPAPIEKTISKARGQPLEPKV | and C-tag (at C- |
| | | YTMGPPREELSSRSVSLTCMINGFYPSDISVEWEKNGK | terminus) |
| | | AEDNYKTTPAVLDSDGSYFLYSKLSVPTSEWQRGDVFT | |
| | | CSVMHEALHNHYTQKSISRSPGKHHHHHHHHEPEA | |
| SEQ ID No: 49 | DR5sh79-115ECDdel- | MEQRGQNAPAASGARKRHGPGPREARGARPGPRVP | |
| | FcRbHisCtag | KTLVLVVAAVLLLVSAESALITQQDLAPQQRAAPQQKR | |
| | | SSPSEGPCLAGQYLSEGNCKPCREGIDYTSHSNHSLDSC | |
| | | ILCTRCDSGEVELSPCTTTRNTVCQCEEGTFREEDSPEM | |
| | | CRKCRTGCPRGMVKVGDCTPWSDIECVHKESGAPSTC | |
| | | SKPTCPPPELLGGPSVFIFPPKPKDTLMISRTPEVTCVVV | |
| | | DVSQDDPEVQFTWYINNEQVRTARPPLREQQFNSTIR | |
| | | VVSTLPIAHQDWLRGKEFKCKVHNKALPAPIEKTISKAR | |
| | | GQPLEPKVYTMGPPREELSSRSVSLTCMINGFYPSDISV | |
| | | EWEKNGKAEDNYKTTPAVLDSDGSYFLYSKLSVPTSEW | |
| | | QRGDVFTCSVMHEALHNHYTQKSISRSPGKHHHHHH | |
| | | HHEPEA | |
| SEQ ID No: 50 | DR5sh139-166ECDdel- | MEQRGQNAPAASGARKRHGPGPREARGARPGPRVP | |
| | FcRbHisCtag | KTLVLVVAAVLLLVSAESALITQQDLAPQQRAAPQQKR | |
| | | SSPSEGLCPPGHHISEDGRDCISCKYGQDYSTHWNDLL | |
| | | FCLRCTRCDSGEVELSPCTTTRNTVCQCKPGTFEDKDS | |
| | | PEICQSCSNCTDGEEEVGDCTPWSDIECVHKESGAPST | |
| | | CSKPTCPPPELLGGPSVFIFPPKPKDTLMISRTPEVTCVV | |
| | | VDVSQDDPEVQFTWYINNEQVRTARPPLREQQFNSTI | |
| | | RVVSTLPIAHQDWLRGKEFKCKVHNKALPAPIEKTISKA | |
| | | RGQPLEPKVYTMGPPREELSSRSVSLTCMINGFYPSDIS | |
| | | VEWEKNGKAEDNYKTTPAVLDSDGSYFLYSKLSVPTSE | |
| | | WQRGDVFTCSVMHEALHNHYTQKSISRSPGKHHHHH | |
| | | HHHEPEA | |

The present invention is further illustrated by the following Examples which should not be construed as further limiting.

### EXAMPLES

### Example 1: Antibody and Antigen Constructs

### Expression Constructs for DR5 Antigen

Codon-optimized constructs for membrane expression of the short isoform of human DR5 with death domain loss-of-function mutation K386N (SEQ ID No: 44; based on UniprotKB/Swiss-Prot O14763-2), and cynomolgus monkey DR5 with deletion of amino acids 185-213 and death domain loss-of-function mutation K420N (SEQ ID No: 46; based on NCBI accession number XP_005562887.1), were generated. The constructs were cloned in the mammalian expression vector pcDNA3.3 (Invitrogen). DR5 expression constructs were transiently transfected in Freestyle CHO-S cells (Life technologies, Cat no R80007), using the FreeStyle MAX Reagent (Invitrogen by Life technologies, Cat no 16447-100), as described by the manufacturer. Transfected cells were stored in liquid nitrogen.

Codon-optimized chimeric human/mouse DR5 construct for soluble extracellular domain (ECD) of DR5 with a C-terminal tag was generated in which the sequence stretches 79-115 or 139-166 in human DR5 (SEQ ID No: 42) were replaced by the corresponding mouse DR5 sequence (SEQ ID No: 47): DR5sh79-115ECDdelHis (SEQ ID No: 50) and DR5sh139-166ECDdelHis (SEQ ID No:49), respectively (numbers referring to the human sequence). The construct contained suitable restriction sites for cloning and an optimal Kozak (GCCGCCACC) sequence and was cloned in the mammalian expression vector pcDNA3.3 (Invitrogen).

### Expression Constructs for Antibodies

For antibody expression the VH and VL sequences were cloned in expression vectors (pcDNA3.3) containing the relevant constant HC and LC regions. Desired mutations were introduced either by gene synthesis or site directed mutagenesis.

HexaBody-DR5/DR5 is a 1:1 mixture of IgG1-hDR5-01-G56T-E430G and IgG1-hDR5-05-E430G, two non-competing humanized anti-human DR5 IgG1 antibodies (WO14009358; WO17093448; US20170260281) with an E430G hexamerization-enhancing mutation in their Fc domains (Diebolder et al., Science 2014; de Jong et al., PLoS Biol. 2016).

In some of the examples gp120-specific human IgG1 antibody IgG1-b12 or IgG1-b12-E430G was used as negative (isotype) control (Barbas et al., J Mol Biol. 1993 Apr. 5; 230(3):812-23).

### Transient Expression

Antibodies were expressed as IgG1,κ by GeneArt or in house by Genmab BV. At Genmab, plasmid DNA mixtures encoding both heavy and light chains of antibodies were transiently transfected in Expi293F cells (Life technologies) using 293fectin (Life technologies), essentially as described by Vink et al. (Vink et al., Methods, 65 (1), 5-10 2014).

Membrane proteins were expressed in Freestyle CHO-S cells (Life technologies), using the freestyle Max reagent, as described by the manufacturer.

### Purification and Analysis of Proteins

Antibodies were purified by immobilized protein A chromatography. His-tagged recombinant protein was purified by immobilized metal affinity chromatography. Protein batches were quality checked (QC) by a number of assays applicable to the protein, such as binding, sodium dodecyl sulphate polyacrylamide gel electrophoresis (SDS-PAGE), size exclusion chromatography (SEC), mass spectrometry (MS) and measurement of endotoxin levels.

### Example 1: DR5 Antibody Binding to Transfected CHO-S Cells

Frozen transfected CHO-S cells were quickly thawed at 37° C. and suspended in 10 mL medium (RPMI 1640 with 25mM Hepes and L-Glutamine [Lonza, Cat no BE12-115F]+50 Units penicillin/50 Units streptomicin [Pen/Strep; Lonza, Cat no DE17-603E]+10% heat-inactivated Donor Bovine Serum with Iron [DBSI; Life Technologies, Cat no 10371-029]). Cells were washed with PBS and resuspended in FACS buffer (PBS+0.1% w/v bovine serum albumin [BSA; Roche, Cat no 10735086001]+0.02% w/v sodium azide) at a concentration of 1.0×10⁶ cells/mL. 100 μL cell suspension samples (100,000 or 50,000 cells per well) were seeded in 96-well ps plates (Greiner Bio-One, Cat no 650101) and pelleted by centrifugation at 300×g for 3 minutes at 4° C. 25 μL of dilution antibody preparation series (0-20 μg/mL final antibody concentrations in 6-fold dilutions) was added and incubated for 30 minutes at 4° C. Next, cells were washed once with 150 μL FACS buffer and incubated with 50 μL secondary antibody R-phycoerythrin (R-PE)-conjugated goat-anti-human IgG F(ab′)₂ (Jackson ImmunoResearch, Cat no 109-116-098; 1/100) for 30 minutes at 4° C. protected from light. Cells were washed once with 150 μL FACS buffer, resuspended in 50 μL or 100 μL FACS buffer, and antibody binding was analysed by flow cytometry on a BD LRSFFortessa cell analyzer (BD Biosciences) by recording 10,000 events. Transfection efficacy for the CHO-S cells was not 100%; therefore the geometric mean fluorescence intensity (FI) of the PE positive population was determined. In case the PE positive population could no longer be discriminated from the negative population geometric mean Fl from all cells was determined. Binding curves were analyzed using non-linear regression analysis (sigmoidal dose-response with variable slope) using GraphPad Prism software.

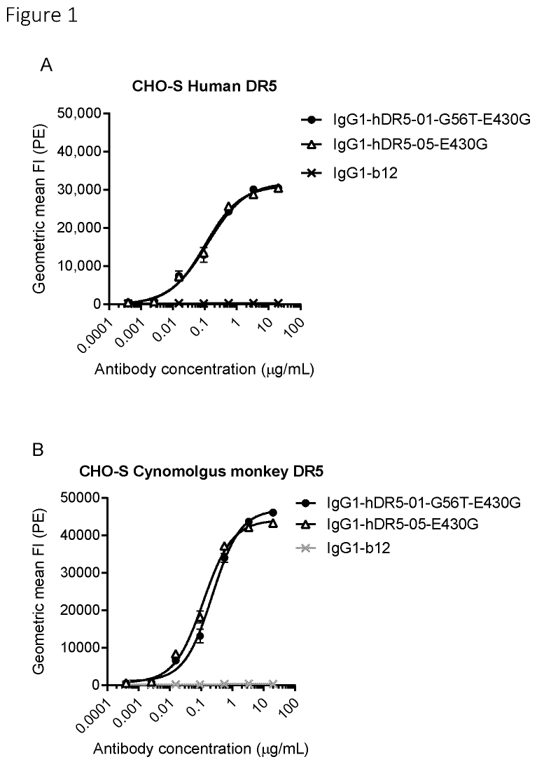

IgG1-hDR5-01-G56T-E430G and IgG1-hDR5-05-E430G showed similar dose-dependent binding to CHO-S cells expressing human and cynomolgus monkey DR5, with apparent affinities (EC50) in the high picomolar-low nanomolar range (FIG. 1, Table 3).

TABLE 3
EC50 values for antibody binding to human and cynomolgus monkey DR5. Antibody binding
was tested by flow cytometry using CHO-S cells expressing human DR5 and cynomolgus monkey
DR5. EC50 values were calculated from the dose response-curve using GraphPad Prism software.
Average EC50 values were calculated from four independent experiments.

| | EC50 for human DR5 | | EC50 for cynomolgus monkey DR5 | |
|---|---|---|---|---|
| | Average (n = 4) | | Average (n = 4) | |
| Antibody | μg/mL (SD) | nM (SD) | μg/mL (SD) | nM (SD) |
| IgG1-hDR5-01-G56T-E430G | 0.13 (0.03) | 0.87 (0.23) | 0.27 (0.18) | 1.77 (1.16) |
| IgG1-hDR5-05-E430G | 0.12 (0.03) | 0.83 (0.18) | 0.17 (0.08) | 1.16 (0.56) |

### Example 2: Nonclinical Pharmacological Active Dose

### In Vitro Viability Assays Using Cell Lines

The in vitro cytotoxicity of the mixture of IgG1-hDR5-01-G56T-E430G+IgG1-hDR5-05-E430G was determined in viability assays in various human cancer cell lines (Table 4). COLO 205 cancer cells were harvested by pooling the culture supernatant containing non-adherent cells and trypsinized adherent cells. The other cell lines were harvested by trypsinization. For trypsinization, adherent cells were incubated with Trypsin-EDTA (Gibco, Cat no 15400-054) diluted in PBS (B.Braun; Cat no 3623140) to a final concentration of 0.05% Trypsin for 2 minutes at 37° C. and passed through a cell strainer. Cells were pelleted by centrifugation for 5 minutes at 1,200 rpm and resuspended at a concentration of 0.5x105 cells/mL in culture medium.

TABLE 4
Cell lines used in viability assays in vitro

| | | Company, | |
|---|---|---|---|
| Cell line | Origin | Cat no | Culture medium |
| A375 | Melanoma | ATCC | DMEM 4.5 g/L Glucose without L-Glutamine with HEPES |
| | | CRL-1619 | (Lonza, Cat no BE12-709F) + 2 mM L-glutamine (Lonza, Cat |
| | | | no BE17-605F) + 10% heat-inactivated Donor Bovine Serum |
| | | | with Iron (DBSI; Life Technologies Cat no 10371-029) + 50 |
| | | | Units/mL penicillin/50 Units/mL streptomicin (Pen/Strep; |
| | | | Lonza Cat no DE17-603E) |
| Bx-PC-3 | Pancreatic | ATCC | RPMI 1640 with 25 mM Hepes and L-Glutamine (Lonza, Cat |
| | cancer | CRL-1687 | no BE12-115F) + 10% heat-inactivated DBSI + Pen/Strep |
| HPAF-II | Pancreatic | ATCC | EMEM with low sodium bicarbonate (ATCC, Cat no 30-2003) + |
| | cancer | CRL-1997 | 10% heat-inactivated DBSI + Pen/Strep |
| PANC-I | Pancreatic | ATCC | DMEM 4.5 g/L Glucose without L-Glutamine and HEPES |
| | cancer | CRL-1469 | (Lonza) + 2 mM L-glutamine (Lonza) + 1 mM Sodium |
| | | | Pyruvate (Lonza, Cat no BE13-115E) + 10% heat-inactivated |
| | | | DBSI + Pen/Strep |
| COLO 205 | Colorectal | ATCC | RPMI 1640 with 25 mM Hepes and L-Glutamine (Lonza) + |
| | cancer | CCL-222 | 10% heat-inactivated DBSI + Pen/Strep |
| HCT 116 | Colorectal | ATCC | McCoy's 5A medium with L-Glutamine and HEPES (Lonza, |
| | cancer | CCL-247 | Cat no BE12-168F) + 10% heat-inactivated DBSI + Pen/Strep |
| HCT-15 | Colorectal | ATCC | RPMI 1640 with 25 mM Hepes and L-Glutamine (Lonza) + |
| | cancer | CCL-225 | 10% heat-inactivated DBSI + Pen/Strep |
| HT-29 | Colorectal | ATCC | McCoy's 5A medium with L-Glutamine and HEPES (Lonza) + |
| | cancer | HTB-38 | 10% heat-inactivated DBSI + Pen/Strep |
| SW480 | Colorectal | ATCC | RPMI 1640 with 25 mM Hepes and L-Glutamine (Lonza) + |
| | cancer | CCL-228 | 10% heat-inactivated DBSI + Pen/Strep |
| A549 | Lung cancer - | ATCC | Ham's F-12K Medium with low sodium bicarbonate (ATCC, |
| | NSCLC | CCL-185 | Cat no 30-2004) + 2 mM L-glutamine (Lonza) + 10% heat- |
| | | | inactivated DBSI + Pen/Strep |
| SK-MES-1 | Lung cancer - | ATCC | EMEM with low sodium bicarbonate (ATCC) + 10% heat- |
| | NSCLC | HTB-58 | inactivated DBSI + Pen/Strep |
| SNU-5 | Gastric | ATCC | IMEM with HEPES and L-glutamine (Lonza, Cat no BE12- |
| | cancer | CRL-5973 | 722F) + 10% heat-inactivated DBSI + Pen/Strep |

100 μL of single cell suspensions (5,000 cells per well) were seeded in polystyrene 96-well flat-bottom plates (Greiner Bio-One, Cat no 655180) and allowed to adhere overnight at 37° C. The following day, 50 μL antibody samples (0.002-133 nM final concentrations in 4-fold dilutions) were added to the adherent cells and incubated for 3 days at 37° C. As a positive control in all viability assays, cells were incubated with 5 μM staurosporine (Sigma Aldrich, Cat no S6942), and untreated cells were included as the negative control. The viability of the cultured cells was determined in a CellTiter-Glo Luminescent Cell Viability Assay (Promega, Cat no G7571) that quantifies the presence of ATP, which is an indicator of metabolically active cells. From the kit, 15 μL Luciferin Solution Reagent was added to each well of the viability assay plate. Next, plates were incubated for 1.5 hours at 37° C. 100 μL supernatant was transferred to a white OptiPlate-96 (Perkin Elmer, Cat no 6005299) and luminescence was measured on an EnVision Multilabel Reader (PerkinElmer). Data were analyzed and plotted using non-linear regression (sigmoidal dose-response with variable slope) using GraphPad Prism software. The percentage viable cells was calculated using the following formula: % viable cells=[(luminescence antibody sample−luminescence staurosporine sample)/(luminescence no antibody sample−luminescence staurosporine sample)]×100.

The mixture of IgG1-hDR5-01-G56T-E430G and IgG1-hDR5-05-E430G induced dose-dependent cytotoxicity reaching more than 40% maximal inhibition of viability in seven of the twelve different cell lines tested. Average values for IC20 and maximal inhibition from at least three independent experiments are presented in Table 5.

TABLE 5
Average IC20 and percentage of maximal growth inhibition
from three-day viability assays with IgG1-hDR5-
01-G56T-E430G + IgG1-hDR5-05-E430G performed
with cell lines showing >40% max inhibition

| | | |
|---|---|---|
| Cancer | Average IC20 | Max inhibition |

| Cell line | subtype | nM | μg/mL | % |
|---|---|---|---|---|
| A375 | Melanoma | 0.445 | 0.067 | 54.6 |
| BxPC-3 | Pancreatic | 0.677 | 0.102 | 89.4 |
| PANC-1 | Pancreatic | 0.554 | 0.083 | 61.0 |
| COLO 205 | CRC | 0.103 | 0.015 | 99.6 |
| HCT-15 | CRC | 0.692 | 0.104 | 66.7 |
| SK-MES-1 | NSCLC | 0.572 | 0.086 | 76.6 |
| SNU-5 | Gastric | 0.274 | 0.041 | 44.7 |

| | | |
|---|---|---|
| Overall Median | 0.554 | 0.083 |

### In Vivo Anti-Tumor Activity Using CDX Models

The in vivo anti-tumor efficacy of different doses of the mixture of IgG1-hDR5-01-G56T-E430G+IgG1-hDR5-05-E430G was tested in xenograft tumor models derived from established human tumor cell lines (CDX) representing different solid tumor indications (Table 6). In these studies, a mixture of human/mouse chimeric antibodies containing a K409R or F405L mutation (IgG1-DR5-01-K409R-E430G+

IgG1-DR5-05-F405L-E430G) was used as surrogate for IgG1-hDR5-01-G56T-E430G+IgG1-hDR5-05-E430G, which is known to show functional comparability. Immunodeficient 7-10 weeks old female CB17-SCID (C.B-17/IcrHan® Hsd-Prkdc^{scid}, Harlan), BALB/c athymic nude mice (Shanghai Laboratory Animal Center, China) or NOD/SCID mice (Beijing HFK Bioscience) were used in the CDX studies. Earmarks were placed for mouse identification. Tumors were induced by subcutaneous injection of 100-200 μL tumor cell suspension containing three to ten million cells in the flank of the mouse.

TABLE 6
CDX models in vivo

| | | | No of cells | Antibody doses (mg/kg) | |
|---|---|---|---|---|---|
| Cell line | Cancer origin | Source | inoculated¹ | and frequency | Route |
| COLO 205 | Colorectal | ATCC | 3 × 10⁶ | 0.1-0.5-2.0 (single dose) | IV |
| HCT-15 | Colorectal | Crown Bioscience | 5 × 10⁶ | 0.5-2.0-10 (Q7Dx2) | IV |
| SW480 | Colorectal | Crown Bioscience | 1 × 10⁷ | 0.5-2.0-10 (Q7Dx2) | IV |
| BxPC-3 | Pancreatic | ATCC | 5 × 10⁶ | 0.5-2.0-10 Q7Dx2) | IV |
| A375 | Melanoma | ATCC | 5 × 10⁶ | 0.5-2.0-10 (Q7Dx2) | IV |
| SNU-5 | Gastric | Crown Bioscience | 1 × 10⁷ | 0.5-2.0-10 (Q7Dx2) | IV |
| SK-MES-1 | Squamous lung | Crown Bioscience | 5 × 10⁶ | 0.5-2.0-10 (Q7Dx2) | IV |

¹All cell lines were harvested in log-phase (at a confluence of approximately 70%).

Mice were divided into groups of 6-8 mice each, with equal tumor size distribution (average and variance). Mice were injected intravenously (IV) with 0.1 mL test solution per mouse, according to the specific schedules mentioned in Table 6. In most studies, the body weight of the mice was monitored twice weekly, including on the day of treatment. Mice were observed at least twice a week for clinical signs of illness. Tumor volumes were measured at least twice a week using a digital caliper (PLEXX). Tumor volumes (mm3) were calculated as follows: tumor volume=0.52×(length)×(width)². Statistical differences in median tumor volumes were compared between treatment groups using Mann Whitney test on the last day treatment groups were complete using graphpad prism software. Mantel-Cox analysis of Kaplan-Meier curves was performed to analyze statistical differences in progression-free survival time with a general tumor size cut-off of 500 mm³ using IBM SPSS statistics.

The experiments were ended for individual mice when the tumor size exceeded 1.5 cm³, the tumor showed ulceration, in case of serious clinical illness, when the tumor growth blocked the movement of the mouse, or when tumor growth assessment had been completed.

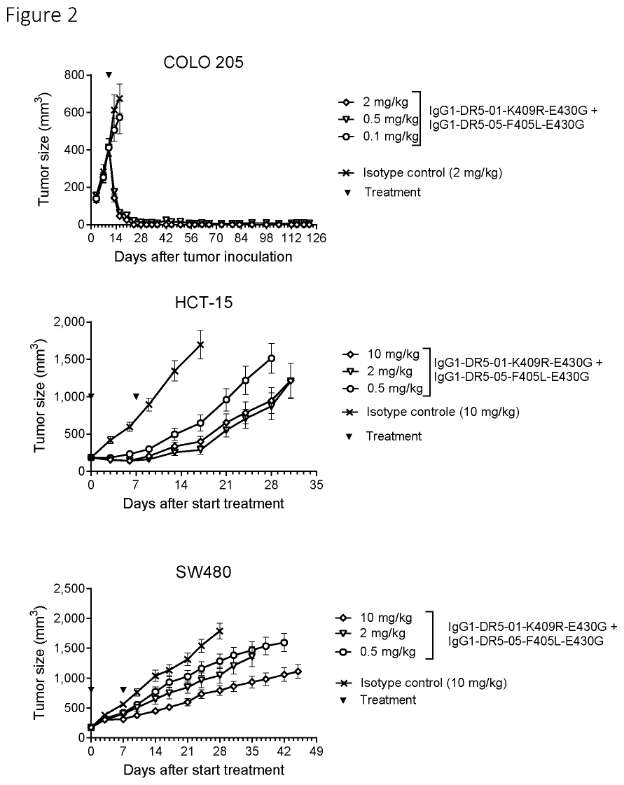

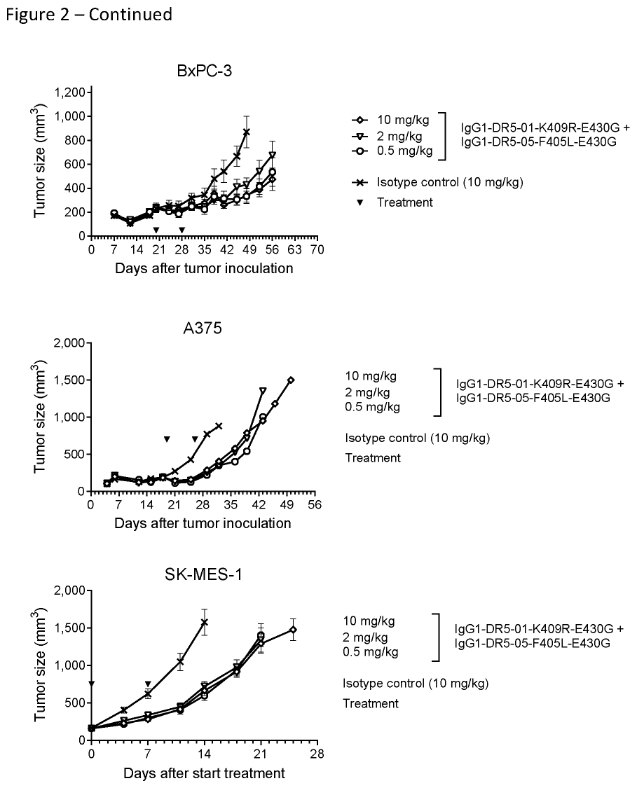

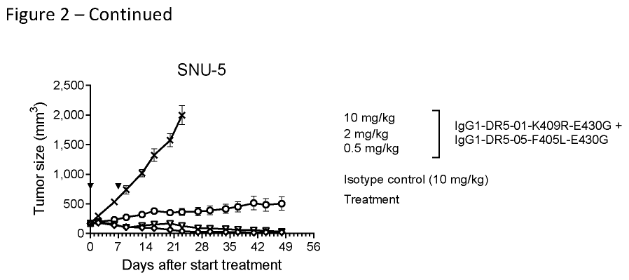

The mixture of IgG1-DR5-01-K409R-E430G+IgG1-DR5-05-F405L-E430G inhibited tumor growth at 0.5 mg/kg and 2 mg/kg in the COLO 205 CDX model, and at 0.5 mg/kg, 2 mg/kg and 10 mg/kg in the HCT-15, SW480, BxPC-3A375, SK-MES-1 and SNU-5 CDX models (FIG. 2).

### In Vivo Anti-Tumor-Activity Using PDX Models

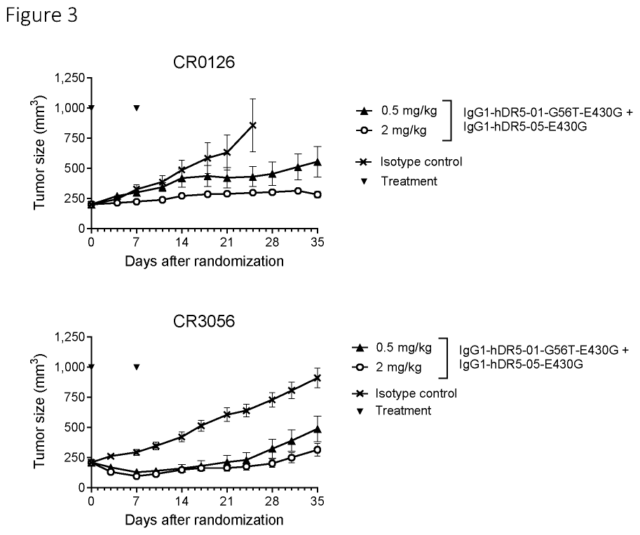

Immunodeficient 8-13 weeks old female BALB/c athymic nude mice (Beijing HFK Bio-Technology Co. Ltd.) or nu/nu mice (Vital River Laboratories Research Models and Services) were inoculated subcutaneously at the right flank with one tumor fragment (2-3 mm diameter) derived from colorectal cancer PDX model CR0126 or CR3056 at CrownBio, Beijing, China. Earmarks were placed for mouse identification. Tumor volumes were measured at least twice per week using a digital caliper (PLEXX). Tumor volumes (mm³) were calculated as follows: tumor volume=0.5×(length)×(width)². When the mean tumor size reached ˜200 mm³, mice were divided into groups of 8 mice each, with equal tumor size distribution (average and variance). Mice were treated QW×2 by IV injection of 10 μL IgG1-DR5-01-G56T-E430G+IgG1-DR5-05-E430G per gram body weight: 0.2 mg/mL (2 mg/kg) or 0.05 mg/mL (0.5 mg/kg). For each model, statistical analysis was performed on the last day that all groups were intact. IgG1-DR5-01-G56T-E430G+IgG1-DR5-05-E430G showed significant tumor growth inhibition at 2 mg/kg (Mann-Whitney test; p<0.0379) in the CR0126 model, and at both 2 mg/kg and 0.5 mg/kg Hx-DR5-01/05 (Mann-Whitney test; p<0.0003 and p<0.0379 respectively) in the CR3056 model (FIG. 3).

### Example 3: PK in Cynomolgus Monkey

### Single Dose Study of the Mixture of IgG1-hDR5-01-G56T-E430G+IgG1-hDR5-05-E430G by Intravenous Infusion in Cynomolgus Monkeys

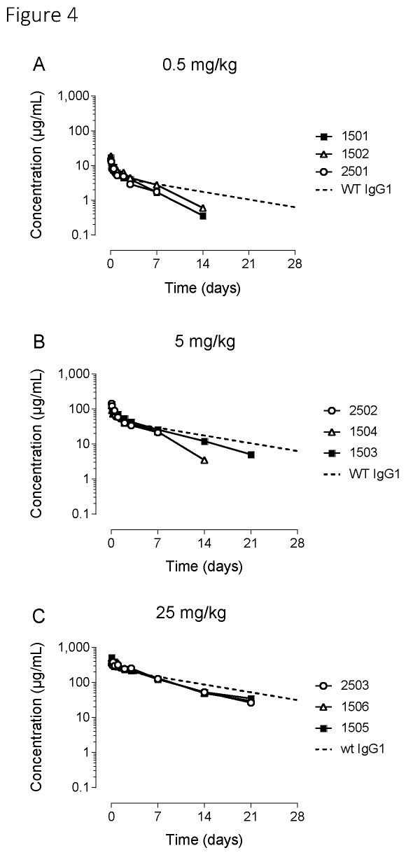

Human antibody plasma concentration profiles were measured using a generic IgG PK electrochemiluminescence immunoassay (ECLIA) after intravenous single dose infusion of the mixture of IgG1-hDR5-01-G56T-E430G+IgG1-hDR5-05-E430G at dose levels of 0.5, 5 and 25 mg/kg (infusion time 30 min, n=3 females, time points post-dose: 1, 3, 6, 12, 24 hours, 2, 3, 7, 14 and 21 days). In short, IgG1-hDR5-01-G56T-E430G+IgG1-hDR5-05-E430G were captured on a coat of monoclonal anti Human IgG (non-cross reactive with Cynomolgus IgG) antibody. Captured IgG was detected by using another monoclonal anti Human IgG (non-cross reactive with Cynomolgus IgG) conjugated to SULFO-TAG. This complex was visualized using an ECL imager. Plasma concentration-time profiles were consistent with the intravenous dose route of the test item (FIG. 4). Individual estimates of the half-life (T_{1/2}) ranged from 3.16 to 7.57 days. Individual clearance values ranged from 8.98 to 14.2 mL/day/kg. Individual volumes of distribution values ranged from 52.4 to 98.8 mL/kg, indicating a limited distribution of the test item beyond the circulation. Based on nAUC_{0-∞}, a more or less proportional increase in exposure was seen with increasing dose. Based on nCₘₐₓ, a proportional increase in exposure was seen up to a dose of 5 mg/kg, whereas between 5 and 25 mg/kg a less than proportional increase in exposure was observed.

Individual plasma concentration profiles are shown in FIG. 4 and group mean toxicokinetic parameters are shown in Table 7.

TABLE 7
Mean PK parameters in single IV dose study of IgG1-hDR5-
01-G56T-E430G + IgG1-hDR5-05-E430G in cynomolgus monkeys

| Dose | | Cₘₐₓ | nCₘₐₓ | | AUC_{0-∞} | nAUC_{0-∞} | CL | Vd |
|---|---|---|---|---|---|---|---|---|
| (mg · | Tₘₐₓ | (μg · | (μg · mL⁻¹/ | T_{1/2} | (μg · | (μg · day · mL⁻¹/ | (mL · | (mL · |
| kg⁻¹) | (day) | mL⁻¹) | mg · kg⁻¹) | (day) | day/· mL⁻¹) | mg · kg⁻¹) | day⁻¹/· kg⁻¹) | kg⁻¹) |
| 0.5 | 0.0417 | 16.7 | 33.5 | 3.48 | 40.3 | 80.6 | 12.6 | 63.5 |
| 5 | 0.0417 | 135.0 | 27.0 | 4.59 | 452.6 | 90.4 | 11.4 | 72.6 |
| 25 | 0.14 | 470.3 | 18.8 | 6.93 | 2748 | 109.9 | 9.10 | 91.0 |

### Dose Range Finding Study of the Mixture of IgG1-hDR5-01-G56T-E430G+IgG1-hDR5-05-E430G by Intravenous Infusion in Cynomolgus Monkeys

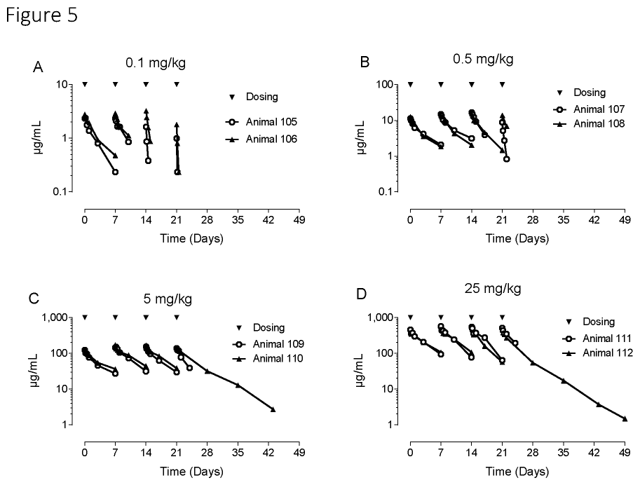

Human antibody plasma concentration profiles were measured using a generic IgG PK ECLIA after IV single dose (n=1) or repeated dose (1Q4×4, n=2) infusion of the mixture of IgG1-hDR5-01-G56T-E430G+IgG1-hDR5-05-E430G at dose levels of 0.1, 0.5, 5 and 25 mg/kg (infusion time 30 min, sampling time points pre-dose and 0.5, 4, 12, 24 and 72 hours post-dose). Plasma concentration-time profiles were consistent with a wild type human IgG1 clearance in cynomolgus monkeys, with no indications of target-mediated clearance (FIG. 5). Based on nCₘₐₓ and nAUC_{0-∞}, a proportional increase in exposure was seen with increasing dose; based on nCmax a less than proportional increase in exposure was observed between the mid- and high-dose groups, only in the single-dose treated groups.

### Repeat-Dose Toxicity Study with 5 Repeated Weekly IV Infusions of the Mixture of IgG1-hDR5-01-G56T-E430G+IgG1-hDR5-05-E430G in Cynomolgus Monkeys

Four groups of five male and five female cynomolgus monkeys each received once-weekly 30 minute intravenous infusions, on Days 1, 8, 15, 22 and 29 of the dosing phase, as follows:

| Group number | Group description | Dose level (mg/kg) |
|---|---|---|
| 1 | Control | 0 (vehicle) |
| 2 | Low | 2 |
| 3 | Intermediate | 10 |
| 4 | High | 50 |

For Days 1, 8, 15, 22 and 29, blood samples were taken from each animal pre-dose and 0.5, 4, 12, 24 and 72 hours after the end of infusion. Additional blood samples were taken from each recovery animal (two males and two females per dose group) on Days 36, 43, 50 and 57.

Concentrations of IgG1-hDR5-01-G56T-E430G in cynomolgus monkey plasma were determined using an electrochemiluminescence immunoassay (ECLIA). In this assay, IgG1-hDR5-01-G56T-E430G is captured with coating antigen DR5sh79-115ECDdelHis (SEQ ID NO 48). Captured IgG1-hDR5-01-G56T-E430G was detected by a monoclonal anti Human IgG (non-cross reactive with Cynomolgus IgG) antibody conjugated to SULFO-TAG. The complex was visualized using an ECL imager. This ECLIA detects only IgG1-hDR5-01-G56T-E430G, not IgG1-hDR5-05-E430G.

Concentrations of IgG1-hDR5-05-E430G in cynomolgus monkey plasma were determined using an ECLIA method. In this assay, IgG1-hDR5-05-E430G is captured with coating antigen DR5sh139-166ECDdelHis (SEQ ID NO 49). Captured IgG1-hDR5-05-E430G was detected by a monoclonal anti Human IgG (non-cross reactive with Cynomolgus IgG) antibody conjugated to SULFO-TAG. The complex was visualized using an ECL imager. This ECLIA detects only IgG1-hDR5-05-E430G, not IgG1-hDR5-01-G56T-E430G.

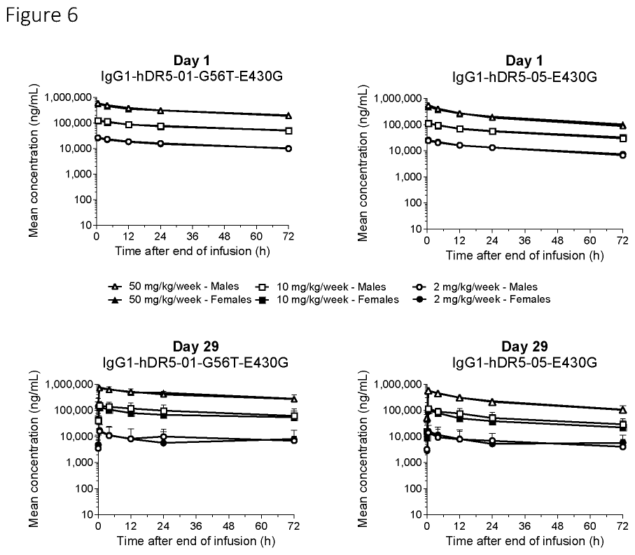

FIG. 6 shows the mean plasma concentration of IgG1-hDR5-01-G56T-E430G and IgG1-hDR5-05-E430G on dosing days 1 and 29. On each sampling occasion, plasma concentrations of both IgG1-hDR5-01-G56T-E430G and IgG1-hDR5-05-E430G were generally at a maximum at the first sampling time point, 1 hour after the start of infusion (0.5 hours after the end of infusion). Clearance of both IgG1-hDR5-01-G56T-E430G and IgG1-hDR5-05-E430G from the systemic circulation was incomplete within the dosing interval after each of the first two once-weekly doses. The extent of clearance of both IgG1-hDR5-01-G56T-E430G and IgG1-hDR5-05-E430G increased following subsequent weekly doses, particularly at the 2 mg/kg/week dose level. On Day 1, where calculable, total serum clearance was between 0.649 and 1.23 mL/h/kg for IgG1-hDR5-01-G56T-E430G and between 0.936 and 1.65 mL/h/kg for IgG1-hDR5-05-E430G. On subsequent sampling occasions, mean Clₛₛ values tended to increase with time. The most rapid clearance tended to occur in the animals that were anti-drug antibody (ADA)-positive and gave the highest ADA responses.

On Day 1, where calculable, elimination half-lives (t_{1/2}) were between 83.1 and 103 hours for IgG1-hDR5-01-G56T-E430G and between 54.5 and 95.4 hours for IgG1-hDR5-05-E430G. On Day 8, where calculable, t_{1/2} was between 26.4 and 103 hours for IgG1-hDR5-01-G56T-E430G and between 27.3 and 89.1 hours for IgG1-hDR5-05-E430G. After subsequent weekly doses, the mean half-life for both compounds tended to decrease, particularly at the low and intermediate dose levels, with greater inter-animal variability.

The observed increases in measures of plasma exposure (Cₘₐₓ and AUC₍₀₋ₜ₎) to both IgG1-hDR5-01-G56T-E430G and IgG1-hDR5-05-E430G following once-weekly intravenous administration were approximately proportional to the increases in dose level. These data suggest that there was no saturation of the clearance of either compound at the higher dose levels.

### Example 4: Human Equivalent Dose

For safety reasons, a lower first in human (FIH) starting dose than the no observed adverse events level (NOAEL)/highest non severely toxic dose (HNSTD)-based maximum recommended starting dose (MRSD) of the mixture of IgG1-hDR5-01-G56T-E430G+IgG1-hDR5-05-E430G of 8.3 mg/kg was used. A FIH clinical trial starting dose of the mixture of IgG1-hDR5-01-G56T-E430G+IgG1-hDR5-05-E430G of 0.3 mg/kg was used. This dose level was considered to be safe and in the lower end of the potential therapeutically active dose range, based on considerations from nonclinical pharmacology, pharmacokinetic and toxicology studies and a preclinical population PK simulation model (performed by BAST GmbH). For modelling purposes, only cynomolgus monkey PK data from the first dosing cycles were used to avoid confounding effects of anti-drug antibodies that were observed upon subsequent dosings.

The NOAEL and HNSTD in cynomolgus monkey following a 1QWx5 IV dosing of the mixture of IgG1-hDR5-01-G56T-E430G+IgG1-hDR5-05-E430G was determined in the pivotal good laboratory practice (GLP) IV toxicity study to be 50 mg/kg, which was converted to an MRSD of 8.3 mg/kg in human.

| | | MRSDₕᵤₘₐₙ |
|---|---|---|
| NOAEL_{cyno} | HNSTD_{cyno} | (=NOAEL_{cyno}/6) |
| (mg/kg) | (mg/kg) | (mg/kg) |
| 50 | 50 | 8.3 |

The in vivo pharmacologically active dose of the mixture of IgG1-hDR5-01-G56T-E430G+IgG1-hDR5-05-E430G as determined using CDX and PDX mouse models (Example 3) was used for conversion to a human equivalent dose level using a preclinical population PK simulation model (performed by BAST GmbH).

Based on these predictions, a human dose of 0.3 mg/kg of the mixture of IgG1-hDR5-01-G56T-E430G+IgG1-hDR5-05-E430G is considered to correspond to the in vivo murine dose level range of 0.5 mg/kg that induced a partial anti-tumor response in the mouse xenograft models. Therefore, the FIH starting dose of 0.3 mg/kg was considered to be in the lower end of a potential therapeutic dose-range in human patients.

| | In vivo anti-tumor | | |
|---|---|---|---|
| | response in CDX and | Predicted human equivalent dose | |
| | PDX mouse models | (mg/kg) | |
| | (mg/kg) | AUC-based | Cₘₐₓ-based |
| Partial response | 0.5 | 0.2 | 0.32 |
| Full response | 2 | 0.8 | 1.26 |

IC20 values of the in vitro cytotoxicity of the mixture of IgG1-hDR5-01-G56T-E430G+IgG1-hDR5-05-E430G in various human cancer cell lines were used for conversion to the minimal anticipated biological effect level (MABEL) in humans. The average IC20 values for the cell lines for which more than 40% inhibition of cell viability was observed with the mixture of IgG1-hDR5-01-G56T-E430G+IgG1-hDR5-05-E430G were used to calculate the median IC20 value to be 0.554 nM (0.083 μg/mL) (Example 3, Table 5). Conversion to a corresponding human dose level was performed using the preclinical population PK simulation model (performed by BAST GmbH), resulted in a MABEL dose of 0.0051 mg/kg in human patients.

The MABEL-based starting dose of 0.0051 mg/kg derived from in vitro cytotoxicity studies with the mixture of IgG1-hDR5-01-G56T-E430G+IgG1-hDR5-05-E430G was not considered appropriate for treatment of patients with advanced cancer for the following reasons: 1) he mixture of IgG1-hDR5-01-G56T-E430G+IgG1-hDR5-05-E430G has shown no immune agonistic properties, and 2) no hazard of acute cytokine-releasing activity has been identified with the mixture of IgG1-hDR5-01-G56T-E430G+IgG1-hDR5-05-E430G.

The nonclinical population PK model was also used to simulate the potential plasma concentration of IgG1-hDR5-01-G56T-E430G and IgG1-hDR5-05-E430G after repeated 2-weekly (1Q2W) i.v. treatment of humans at an assumed therapeutically active dose level of 1 mg/kg of the mixture. According to the in vitro pharmacology studies, the predicted plasma concentrations of IgG1-hDR5-01-G56T-E430G and IgG1-hDR5-05-E430G at trough time after a dose of 1 mg/kg of the mixture was considered to be therapeutically active.

### Example 5: PK in Humans

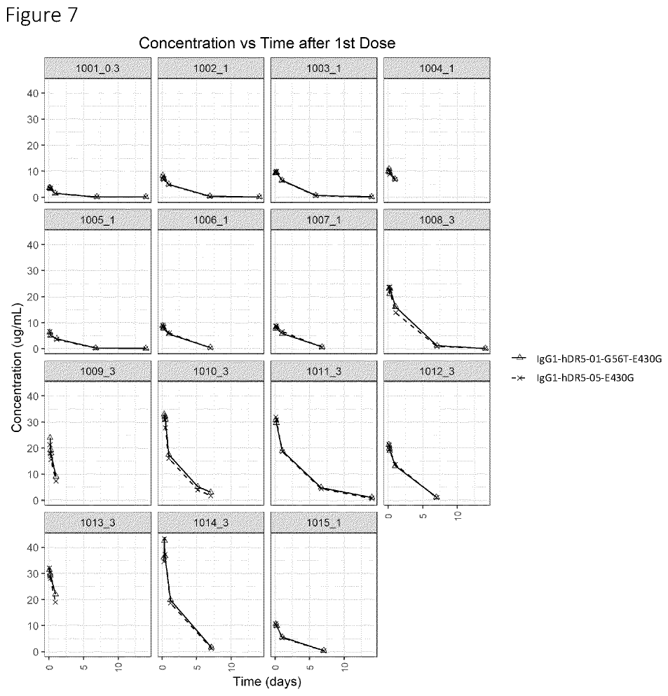

Available PK data from the FIH clinical trial GCT1029-01 with the mixture of IgG1-hDR5-01-G56T-E430G+IgG1-hDR5-05-E430G evaluated after the first dose of 0.3, 1 or 3.0 mg/kg in dose escalation cohorts shows that the PK in human appears to be very similar for the two molecules IgG1-hDR5-01-G56T-E430G and IgG1-hDR5-05-E430G (FIG. 7). In addition, plasma half-life (Tx) was found to be relatively short ranging from 15 to 80 hours. These data indicate that the human PK predictions based on the preclinical PK model as described in Example 5 have underestimated the human clearances of both IgG1-hDR5-01-G56T-E430G and IgG1-hDR5-05-E430G, thereby resulting in Ctrough values of both antibodies below the lower limit of quantification (LLOQ) (based on bi-weekly dosing interval (1Q2W)) and AUC_{0-14 days} below the predicted values. Therefore, the applied bi-weekly dosing regimen may not allow to achieve the best therapeutic index, and an intensified weekly schedule and priming doses have been postulated to be able to increase the dose and thereby obtain higher drug exposures and likely improved anti-tumor efficacy.

### Example 6: Cytotoxicity Of Hexabody-DR5/DR5 in Multiple Myeloma Cells

Bone marrow-derived mononuclear cells (BMNCs) were obtained from MM patients, including newly diagnosed (ND) and relapsed and/or refractory (RR) MM patients. RR MM patients in this analysis had received at least two different prior therapies, including proteasome inhibitors (such as bortezomib and/or carfilzomib) and/or immunomodulatory drugs (such as dexamethasone, prednisone, cyclophosphamide, thalidomide, pomalidomide and/or lenalidomide) and/or therapeutic antibodies (such as CD38-targeting daratumumab or PD-L1-targeting durvalumab), DNA/RNA interfering chemotherapeutic agents (such as bendamustine, melphalan and/or doxorubicin) and/or (autologous) stem cell transplantation (SCT) and/or donor lymphocyte infusion (DLI) and/or any combination thereof, varying between the patients. The capacity of the mixture of IgG1-hDR5-01-G56T-E430G+IgG1-hDR5-05-E430G (HexaBody-DR5/DR5) to induce cytotoxicity in the MM samples was explored in vitro. Cryopreserved BMNCs were thawed, suspended in culture medium (RPMI1640+10% fetal calf serum+1% Penicillin/Streptomycin), washed twice and allowed to recover for minimal 1 h and maximally overnight in culture medium at 37° C. to regain CD138 expression after freeze/thawing. 50 μL samples of the cell suspensions (100,000 cells/well) were added to 96-wells u-bottom plates (Greiner bio-one; Cat no 650180). 50 μL antibody samples (final concentration 20 μg/mL) were added to the cells and incubated for 24 h at 37° C. Plates were centrifuged and cells were resuspended in 100 μL FACS buffer (PBS supplemented with 20% human serum albumin and 0.05% sodium azide) with or without 5 μL counting beads (Flow-Count Fluorospheres; Beckman Coulter; Cat no 7547053). Plates were centrifuged at 2,000 RPM for 5 min and cells were incubated for 15 min at room temperature with 20 μL of an MM lineage marker antibody mixture containing CD138-PE (Beckman Coulter; Cat no A40316; 1:300) and CD38-BV421 (BD Biosciences; Cat no 646851; 1:20) diluted in FACS buffer in presence of the life/death marker 7-AAD (BD Biosciences, Cat no 555816; 1:10). Plates were centrifuged and cells were suspended in 100 μL FACS buffer. Viability of MM cells was determined by flow cytometry on an LSRFortessa or FACSCelesta and the live MM cell subset (7AAD^{neg}/CD138^{pos}/CD38^{pos}) was identified. Percentage inhibition of viability (cell killing) in the MM cell populations was then calculated as follows: Viability inhibition=100%−[(viable MM cell counts in the test sample/average viable MM cell counts in the negative control samples)×100%] using either the average of duplicate no antibody control samples, the average of duplicate IgG1-b12-E430G isotype control samples, or the average of the no antibody and IgG1-b12-E430G isotype controls.

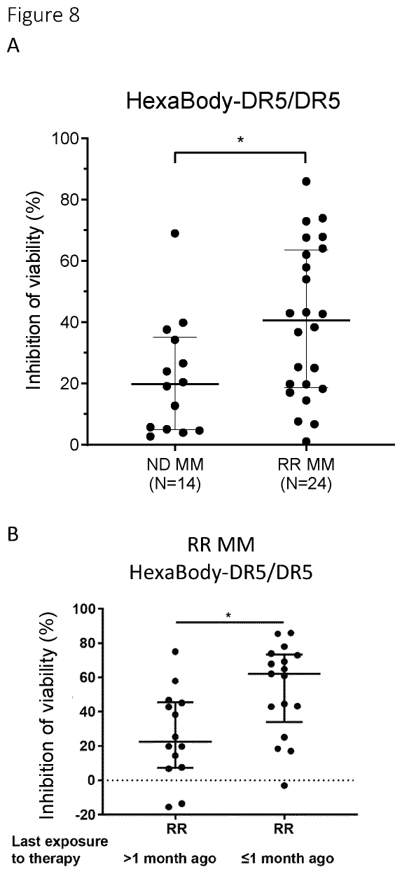

HexaBody-DR5/DR5 induced killing of MM cells, with significantly higher efficiency in samples of patients with relapsed and/or refractory (RR) disease (median lysis: 43%; range 0-86%) compared to newly diagnosed (ND) patient samples (median lysis: 19%; range 3-69%; p<0.01) (FIG. 8A). More specifically, efficacy of HexaBody-DR5/DR5 was significantly higher in samples from patients who were undergoing treatment at the time the BM biopsy was taken, or who had received the last treatment shorter than 1 month before the BM biopsy was obtained (median lysis 62% versus 23%; p<0.01 (FIG. 8B).

### Example 7: Cytotoxicity of Hexabody-DR5/DR5 in Combination with Bortezomib or Lenalidomide in Primary MM Cells

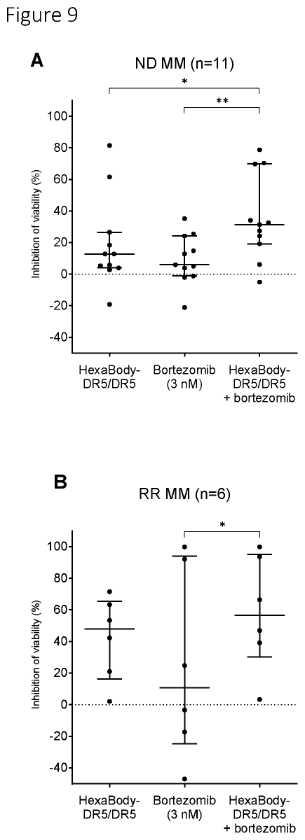

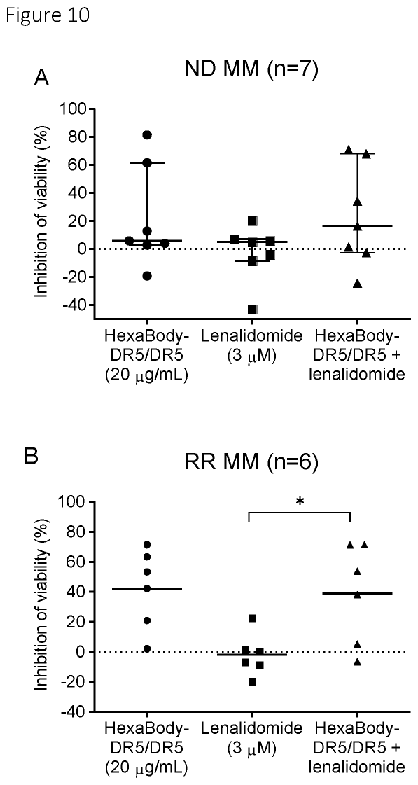

To test if the combination of HexaBody-DR5/DR5 with standard of care therapies used to treat MM could improve the kill of MM cells, HexaBody-DR5/DR5-induced cytotoxicity in MM samples was explored in combination with bortezomib and lenalidomide. Viability assays were performed as described in Example 7, with bortezomib or lenalidomide added simultaneously with HexaBody-DR5/DR5. Combination of 20 μg/mL HexaBody-DR5/DR5 with 3 nM bortezomib resulted in significantly increased cytotoxicity in BMNC samples from ND MM patient samples compared to either monotherapy (FIG. 9A). In BMNC samples from RR MM patients, the combination of HexaBody-DR5/DR5 and bortezomib showed only significantly increased cytotoxicity compared to bortezomib monotherapy, while the increase was not significant compared to HexaBody-DR5/DR5 monotherapy (FIG. 9B). Combination of 20 μg/mL HexaBody-DR5/DR5 with 3 μM lenalidomide showed no increased cytotoxicity in ND MM patient samples compared to either monotherapy (FIG. 10A). In BMNC samples from RR MM patients, the combination of HexaBody-DR5/DR5 and lenalidomide showed significantly increased cytotoxicity compared to lenalidomide monotherapy (Error! Reference source not found.B). Of note, FcγR-positive effector cells were not added to these experiment, so number of FcγR-positive cells is expected to be low. Taken together, no additive effect was observed when HexaBody-DR5/DR5 was combined with lenalidomide in ND and RR MM patient samples in the presence of low numbers of FcγR-positive effector.

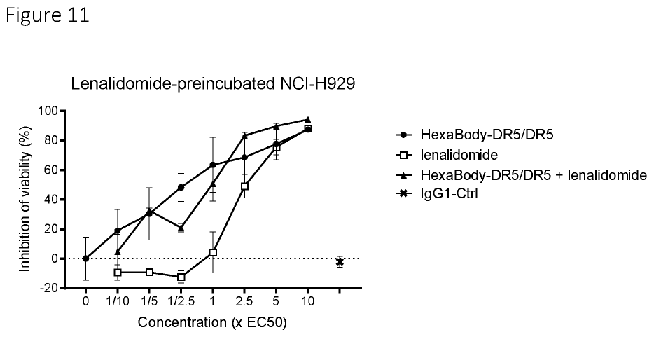

As lenalidomide showed no single agent activity in these 24 h viability assays on primary MM cells ex vivo, the effect of lenalidomide was further assessed in vitro using the MM cell line NCI-H929 that enabled to explore a longer exposure to lenalidomide. However, preincubating NCI-H929 cells for 5 days with 3 μM lenalidomide had no direct effect on HexaBody-DR5/DR5-mediated kill (FIG. 11).

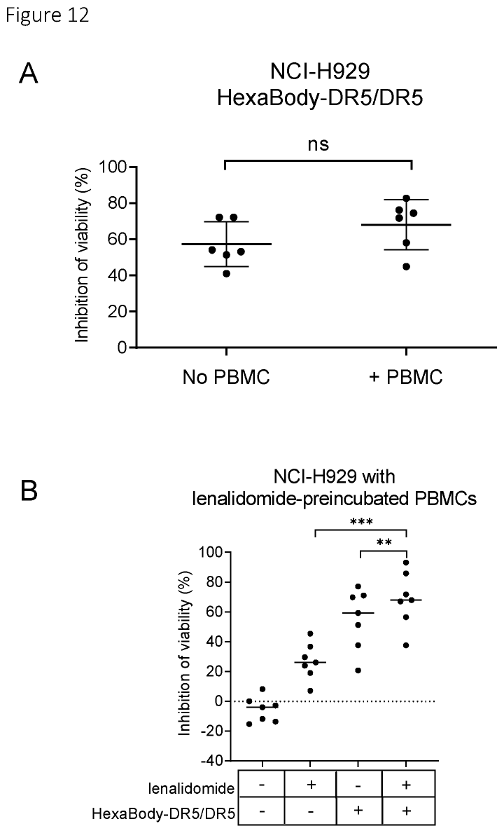

FcγR-mediated crosslinking did not increase HexaBody-DR5/DR5-mediated cytotoxicity in NCI-H929 cells as shown in cytotoxicity assays in absence and presence of healthy donor PBMCs (FIG. 12A). To test possible indirect immunomodulatory effects of lenalidomide on HexaBody-DR5/DR5-mediated killing, PBMCs of healthy donors were preincubated with 3 μM lenalidomide for 5 days. Subsequently, NCI-H929 cells were incubated with 3 μM lenalidomide, lenalidomide-exposed PBMCs and/or 20 μg/mL HexaBody-DR5/DR5 for an additional 24 h. In presence of lenalidomide-preincubated PBMCs, the combination of HexaBody-DR5/DR5 and lenalidomide showed significantly higher kill of the NCI-H929 cells compared to the single agents (FIG. 12B). Together, these data illustrate that lenalidomide enhances HexaBody-DR5/DR5-mediated cytotoxicity predominantly via an immunomodulatory mechanism of action.

1. A method of treating multiple myeloma in a subject, the method comprising administering to a subject in need thereof a first antibody capable of binding DR5 and a second antibody capable of binding DR5, or a pharmaceutically acceptable salt thereof, in combination with an immunomodulatory imide drug.

2. The method of claim 1, wherein the first antibody comprises a variable heavy chain region and a variable light chain region wherein the variable heavy chain region comprises the CDR1, CDR2 and CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

3. The method of claim 1, wherein the second antibody comprises a variable heavy chain region and a variable light chain region wherein the variable heavy chain region comprises the CDR1, CDR2 and CDR3 sequences of SEQ ID Nos: 8, 9, and 10 respectively; and wherein the variable light chain region comprises the CDR1, CDR2 and CDR3 sequences of SEQ ID Nos: 12, RTS, and 13, respectively.

4. The method of any one of claims 1-3, wherein the first and second antibody comprises an Fc region of a human IgG1, wherein the Fc region comprises an E430G mutation of an amino acid position corresponding E430 in human IgG1, wherein the amino acid position is according to the Eu numbering.

5. The method of any one of claims 1 to 4, wherein the first antibody comprises the heavy chain and light chain as set forth in SEQ ID Nos: 17 and 19, respectively.

6. The method of any one of claims 1 to 4, wherein the first antibody comprises the heavy chain and light chain as set forth in SEQ ID Nos: 18 and 19, respectively.

7. The method of any one of claims 1 to 6, wherein the second antibody comprises the heavy chain and light chain as set forth in SEQ ID Nos: 21 and 22, respectively

8. The method of any one of claims 1 to 6, wherein the second antibody comprises the heavy chain and light chain as set forth in SEQ ID Nos: 20 and 22, respectively.

9. The method according to any one of the preceding claims, wherein the immunomodulatory imide drug is thalidomide or a thalidomide analog, e.g. lenalidomide or pomalidomide.

10. The method according to any one of the preceding claims, wherein the immunomodulatory imide drug is selected from the group consisting of thalidomide, lenalidomide and pomalidomide.

11. The method according to any one of the preceding claims, wherein the immunomodulatory imide drug is lenalidomide.

12. The method according to any one of the preceding claims, wherein the multiple myeloma is relapsed and/or refractory multiple myeloma.

13. The method of any one of the preceding claims, wherein the first and second antibody, or a pharmaceutically acceptable salt thereof, is administered simultaneously, separately, or sequentially.

14. The method of any one of the preceding claims, wherein the first and second antibody, or a pharmaceutically acceptable salt thereof, is administered simultaneously.

15. The method of any one of the preceding claims, wherein the first and/or second antibody, or a pharmaceutically acceptable salt thereof, is/are administered by intravenous infusion.

16. The method of any one of the preceding claims, wherein the first and/or second antibody, or a pharmaceutically acceptable salt thereof, is/are administered on day 1 of a 7-days cycle or is/are administered on day 1 of a 14-days cycle.

17. The method of any one of the preceding claims, wherein the first and/or second antibody, or a pharmaceutically acceptable salt thereof, is/are administered on day 1, 8, 15 and 22 of a 28-days cycle or is/are administered on day 1 and 15 of a 28-days cycle.

18. The method according to any one the preceding claims, wherein the first and/or second antibody, or a pharmaceutically acceptable salt thereof, is/are administered at a dose ranging from about 0.05 mg/kg to 18 mg/kg such as from 0.05 mg/kg to 6 mg/kg.

19. The method according to any one the preceding claims, wherein the first and/or second antibody, or a pharmaceutically acceptable salt thereof, is/are administered at a dose of about 0.05 mg/kg, 0.1 mg/kg, 0.15 mg/kg, 0.3 mg/kg, 0.5 mg/kg, 0.6 mg/kg, 1 mg/kg, 1.5 mg/kg, 2.0 mg/kg , 2.25 mg/kg, 3 mg/kg, 4.5 mg/kg, 6 mg/kg, 7.5 mg/kg, 9 mg/kg, 12 mg/kg, 15 mg/kg, 18 mg/kg.

20. The method according to any one of the preceding claims, wherein the first and/or second antibody, or a pharmaceutically acceptable salt thereof, is/are administered to the subject on day 1 of a first 14-day cycle at a dose ranging from about 0.05 mg/kg to 1 mg/kg, such as ranging from about 0.05 mg/kg to 0.3 mg/kg.

21. The method according to any one of the preceding claims, wherein the first and/or second antibody, or a pharmaceutically acceptable salt thereof, is administered to the subject on day 1 of a first and second 14-days cycle at a dose ranging from about 0.05 mg/kg to 1 mg/kg, such as ranging from about 0.05 mg/kg to 0.3 mg/kg.

22. The method according to any one of the preceding claims, wherein when the first and second antibody, or a pharmaceutically acceptable salt thereof, are combined then the total amount of antibody administered is at a dose ranging from about 0.1 mg/kg to 18 mg/kg.

23. The method according to any one of the preceding claims, wherein the first and second antibody, or a pharmaceutically acceptable salt thereof, is administered at about a 1:1 molar ratio.

24. The method according to any one of the preceding claims, wherein the first and second antibody, and optionally the immunomodulatory imide drug, are administered within the same treatment cycle.

25. The method according to any one of the preceding claims, wherein the immunomodulatory imide drug is administered on day 1 to 21 of a 28-days cycle.

26. The method according to any one of the preceding claims, wherein the immunomodulatory imide drug is administered by oral administration.

27. The method according to any one of the preceding claims, wherein the immunomodulatory imide drug is administered at a dose of about 2.5 mg to 25 mg.

28. The method according to any one of the preceding claims, wherein the immunomodulatory imide drug is lenalidomide administered at a dose of about 2.5 mg to 25 mg.

29. A composition comprising a first antibody capable of binding DR5, or a pharmaceutically acceptable salt thereof and second antibody capable of binding DR5, or a pharmaceutically acceptable salt thereof, for use in the treatment of multiple myeloma in combination with an immunomodulatory imide drug.

30. A first antibody or pharmaceutically acceptable salt thereof, capable of binding DR5, for use in the treatment of multiple myeloma in combination with an immunomodulatory imide drug and a second antibody or pharmaceutically acceptable salt thereof, capable of binding DR5.

31. The composition for use according to claim 29, or the first antibody or pharmaceutically acceptable salt thereof for use according to claim 30, wherein the first antibody comprises a variable heavy chain region and a variable light chain region wherein the variable heavy chain region comprises the CDR1, CDR2 and CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

32. The composition for use according to claim 29 or 31, or the first antibody or pharmaceutically acceptable salt thereof for use according to claim 30 or 31, wherein the second antibody comprises a variable heavy chain region and a variable light chain region, wherein the variable heavy chain region comprises the CDR1, CDR2 and CDR3 sequences of SEQ ID Nos: 8, 9, and 10, respectively; and wherein the variable light chain region comprises the CDR1, CDR2 and CDR3 sequences of SEQ ID Nos: 12, RTS, and 13, respectively.

33. The composition for use according to any of claims 29 and 31 to 32, or the first antibody or pharmaceutically acceptable salt thereof for use according to any of claims 30 to 32, wherein the first and second antibody comprises an Fc region of a human IgG1, wherein the Fc region comprises an E430G mutation of an amino acid position corresponding E430 in human IgG1, wherein the amino acid position is according to the Eu numbering.

34. The composition for use according to any of claims 29 and 31 to 33, or the first antibody or pharmaceutically acceptable salt thereof for use according to any of claims 30 to 33, wherein the first antibody comprises the heavy chain and light chain as set forth in SEQ ID Nos: 17 and 19, respectively.

35. The composition for use according to any of claims 29 and 31 to 34, or the first antibody or pharmaceutically acceptable salt thereof for use according to any of claims 30 to 34, wherein the first antibody comprises the heavy chain and light chain as set forth in SEQ ID Nos: 18 and 19, respectively.

36. The composition for use according to any of claims 29 and 31 to 35, or the first antibody or pharmaceutically acceptable salt thereof for use according to any of claims 30 to 35, wherein the second antibody comprises the heavy chain and light chain as set forth in SEQ ID Nos: 20 and 22, respectively.

37. The composition for use according to any of claims 29 and 31 to 36, or the first antibody or pharmaceutically acceptable salt thereof for use according to any of claims 30 to 36, wherein the second antibody comprises the heavy chain and light chain as set forth in SEQ ID Nos: 21 and 22, respectively.

38. The first antibody or pharmaceutically acceptable salt thereof for use according to claim 30, wherein the second antibody comprises a variable heavy chain region and a variable light chain region wherein the variable heavy chain region comprises the CDR1, CDR2 and CDR3 sequences of SEQ ID Nos: 1, 2, and 3 respectively; and wherein the variable light chain region comprises the CDR1, CDR2 and CDR3 sequences of SEQ ID Nos: 5, FAS, and 6, respectively.

39. The first antibody or pharmaceutically acceptable salt thereof for use according to claim 30 or 32, wherein the first antibody comprises a variable heavy chain region and a variable light chain region wherein the variable heavy chain region comprises the CDR1, CDR2 and CDR3 sequences of SEQ ID Nos: 8, 9, and 10, respectively; and wherein the variable light chain region comprises the CDR1, CDR2 and CDR3 sequences of SEQ ID Nos: 12, RTS, and 13, respectively.

40. The first antibody or pharmaceutically acceptable salt thereof for use according to any of claims 30 to 39, wherein the first and second antibody comprises an Fc region of a human IgG1, wherein the Fc region comprises an E430G mutation of an amino acid position corresponding E430 in human IgG1, wherein the amino acid position is according to the Eu numbering.

41. The first antibody or pharmaceutically acceptable salt thereof for use according to any of claims 30 to 40, wherein the second antibody comprises the heavy chain and light chain as set forth in SEQ ID Nos: 17 and 19, respectively.

42. The first antibody or pharmaceutically acceptable salt thereof for use according to any of claims 30 to 40, wherein the second antibody comprises the heavy chain and light chain as set forth in SEQ ID Nos: 18 and 19, respectively.

43. The first antibody or pharmaceutically acceptable salt thereof for use according to any of claims 30 to 42, wherein the first antibody comprises the heavy chain and light chain as set forth in SEQ ID Nos: 20 and 22, respectively.

44. The first antibody or pharmaceutically acceptable salt thereof for use according to any of claims 30 to 42, wherein the first antibody comprises the heavy chain and light chain as set forth in SEQ ID Nos: 21 and 22, respectively.

45. The composition for use according to any of claims 29 and 31 to 37, or the first and second antibody capable of binding DR5, or a pharmaceutically acceptable salt thereof, for use according to any one of claims 30 to 44, wherein the immunomodulatory imide drug is thalidomide or a thalidomide analog, e.g. lenalidomide or pomalidomide.

46. The composition for use according to any of claims 29, 31 to 37 and 45, or the first and second antibody capable of binding DR5, or a pharmaceutically acceptable salt thereof, for use according to any one of claims 30 to 45, wherein the immunomodulatory imide drug is selected from the group consisting of thalidomide, lenalidomide and pomalidomide.

47. The composition for use according to any of claims 29, 31 to 37 and 45 to 46, or the first and second antibody capable of binding DR5, or a pharmaceutically acceptable salt thereof, for use according to any one of claims 30 to 46, wherein the immunomodulatory imide drug is lenalidomide.

48. The composition for use according to any of claims 29, 31 to 37 and 45 to 47, or the first and second antibody capable of binding DR5, or a pharmaceutically acceptable salt thereof, for use according to any one of claims 30 to 47, wherein the immunomodulatory imide drug is administered on day 1 to 21 of a 28-days cycle.

49. The composition for use according to any of claims 29, 31 to 37 and 45 to 48, or the first and second antibody capable of binding DR5, or a pharmaceutically acceptable salt thereof, for use according to any one of claims 30 to 48, wherein the immunomodulatory imide drug is administered by oral administration.

50. The composition for use according to any of claims 29, 31 to 37 and 45 to 49, or the first and second antibody capable of binding DR5, or a pharmaceutically acceptable salt thereof, for use according to any one of claims 30 to 49, wherein the immunomodulatory imide drug is administered at a dose of about 2.5 mg to 25 mg.

51. The composition for use according to any of claims 29, 31 to 37 and 45 to 50, or the first and second antibody capable of binding DR5, or a pharmaceutically acceptable salt thereof, for use according to any one of claims 30 to 50, wherein the immunomodulatory imide drug is lenalidomide administered at a dose of about 2.5 mg to 25 mg.

52. The composition for use according to any of claims 29, 31 to 37 and 45 to 51, or the first and second antibody capable of binding DR5, or a pharmaceutically acceptable salt thereof, for use according to any one of claims 30 to 51, wherein the multiple myeloma is relapsed and/or refractory multiple myeloma.

53. The composition for use according to any of claims 29, 31 to 37 and 45 to 52, or the first and second antibody capable of binding DR5, or a pharmaceutically acceptable salt thereof, for use according to any one of claims 30 to 52, wherein the first and second antibody, or a pharmaceutically acceptable salt thereof, is administered simultaneously, separately, or sequentially.

54. The composition for use according to any of claims 29, 31 to 37 and 45 to 53, or the first and second antibody capable of binding DR5, or a pharmaceutically acceptable salt thereof, for use according to any one of claims 30 to 53, wherein the first and second antibody, or a pharmaceutically acceptable salt thereof, is administered simultaneously.

55. The composition for use according to any of claims 29, 31 to 37 and 45 to 54, or the first and second antibody capable of binding DR5, or a pharmaceutically acceptable salt thereof, for use according to any one of claims 30 to 54, wherein the first and second antibody, or a pharmaceutically acceptable salt thereof, is administered by intravenous infusion.

56. The composition for use according to any of claims 29, 31 to 37 and 45 to 55, or the first and second antibody capable of binding DR5, or a pharmaceutically acceptable salt thereof, for use according to any one of claims 30 to 55, wherein the first and second antibody, or a pharmaceutically acceptable salt thereof, is administered on day 1 of a 7-days cycle or are administered on day 1 of a 14 day cycle.

57. The composition for use according to any of claims 29, 31 to 37 and 45 to 56, or the first and second antibody capable of binding DR5, or a pharmaceutically acceptable salt thereof, for use according to any one of claims 30 to 56, wherein the first and second antibody, or a pharmaceutically acceptable salt thereof, is administered on day 1, 8, 15 and 22 of a 28-days cycle or is/are administered on day 1 and 15 of a 28-days cycle.

58. The composition for use according to any of claims 29, 31 to 37 and 45 to 57, or the first and second antibody capable of binding DR5, or a pharmaceutically acceptable salt thereof, for use according to any one of claims 30 to 57, wherein the composition or the first and second antibody is administered at a dose ranging from about 0.1 mg/kg to 18 mg/kg.

59. The composition for use according to any of claims 29, 31 to 37 and 45 to 58, or the first and second antibody capable of binding DR5, or a pharmaceutically acceptable salt thereof, for use according to any one of claims 30 to 58, wherein the composition or the first and second antibody is administered at a dose of about 0.05 mg/kg, about 0.1 mg/kg, about 0.15 mg/kg, about 0.3 mg/kg, about 0.5 mg/kg, about 0.6 mg/kg, about 1 mg/kg, about 1.5 mg/kg, about 2.0 mg/kg , about 2.25 mg/kg, about 3 mg/kg, about 4.5 mg/kg, about 6 mg/kg, about 7.5 mg/kg, about 9 mg/kg, about 12 mg/kg, about 15 mg/kg, or about 18 mg/kg.

60. The composition for use according to any of claims 29, 31 to 37 and 45 to 59, or the first and second antibody capable of binding DR5, or a pharmaceutically acceptable salt thereof, for use according to any one of claims 30 to 59, wherein the composition or the first and second antibody is administered to the subject on day 1 of a first 7-day cycle or is administered to the subject on day 1 of a first 14-day cycle at a dose ranging from about 0.1 mg/kg to 2 mg/kg, such as ranging from about 0.1 mg/kg to 0.6 mg/kg.

61. The composition for use according to any of claims 29, 31 to 37 and 45 to 60, or the first and second antibody capable of binding DR5, or a pharmaceutically acceptable salt thereof, for use according to any one of claims 30 to 61, wherein the composition or the first and second antibody is administered to the subject on day 1 of a first and second 7-day cycle at a dose ranging from about 0.1 mg/kg to 2 mg/kg, such as ranging from about 0.1 mg/kg to 0.6 mg/kg; or is administered to the subject on day 1 of a first and second 14-day cycle at a dose ranging from about 0.1 mg/kg to 2 mg/kg, such as ranging from about 0.1 mg/kg to 0.6 mg/kg.

62. The composition for use according to any of claims 29, 31 to 37 and 45 to 61, wherein the first and second antibody capable of binding DR5, are at about a 1:1 molar ratio.

63. The first and second antibody capable of binding, or a pharmaceutically acceptable salt thereof, for use according to any one of claims 30 to 61, is administered at about a 1:1 molar ratio.

64. A kit of parts comprising a first antibody capable of binding DR5 and a second antibody capable of binding DR5, or a pharmaceutically acceptable salt thereof; and an immunomodulatory imide drug.

65. The kit of parts according to claim 60, wherein the first and second antibody are as defined in any one of claims 30 to 61 and 63.

66. The kit of part according to any one of claims 60 to 61, wherein the immunomodulatory imide drug is as defined in any one of claims 45 to 47.
